SEQUENCE LISTING (44 sequences)

SEQ ID NO: 1 - protein, 395 aa, Lactobacillus salivarius
  REGION 1..395: note = elongation factor tu
MAKEHYERTKPHVNIGTIGHVDHGKTTLTAAITKVLAEKGLAEASDYASIDAAPEERERGITINTAHVEYETEKRHYAHIDAPGHADYVKNMITGAAQMDGAILVVAATDGPMPQTREHILLARQVGVEYIVVFLNKCDLVDDDELLDLVEMEVRDLLSEYDFPGDDIPVIRGSALKALEGDKDAEAQIMELMDTVDEYIPTPQRPTDKPFLMPVEDVFTITGRGTVASGRIDRGTVKVGDEVEIVGLKDDVVKTTVTGVEMFRKTLDEGEAGDNIGALLRGVDRTQVERGQVLAKPGSIQTHKKFKGEVYVLTKDEGGRHTPFFSNYRPQFYFHTTDVTGVIELPEGVEMVMPGDNVTFTVELIAPVAIEKGLKFTVREGGRTVGAGVVSEIDD

SEQ ID NO: 2 - protein, 450 aa, Lactobacillus salivarius
  REGION 1..450: note = glucose-6-phosphate isomerase
MAHISFDSSNLTKFVNDNELGEMQALVTAVDKELREGTGAGNDFRGFLNLPVDYDKDEFDRIKKAAKKIQSDSEVLVVIGIGGSYLGARAAIDFLHHQFFNLLPAEKRNAPQIFFAGNSISGSYVHDLIELIGDRDFSVNVISKSGTTTEPSIAFRVFKEKLIAKYGVEGARERIYATTDRARGALKTEADAEGYETFVIPDDVGGRFSVLTPVGLLPIAVSGADIDKLMQGAADAREEYSDADLTKNEAYQYAALRNILYRKGYTTEILENYEPTLQYFAEWWKQLMGESEGKDGKGIYPSSANFSTDLHSLGQYIQEGMRNLMETVITVGNPTNDTDIPKEEENLDGLGYLEGKSMNYVNSKAFQGVVLAHTDGNVPNMIVNIPDQTEYTLGYMIYFFEIAVAISGYLNGINPFNQPGVEAYKKNMFALLGRPGYEELTKELQDRLNK

SEQ ID NO: 3 - protein, 400 aa, Lactobacillus salivarius
  REGION 1..400: note = Phosphoglycerate kinase
MAKLIVSDLDVKGKKVLMRVDFNVPIKNGVIGNDNRIVAALPTINYIIEHDGKAILFSHLGRIKKEEDKKELSLRPVAERLAELLNKPVTFVPATRGAQLEDAINNMNDGDVLLVENTRFEDLDGKKESGNDPELGKYWASLGDVFVNDAFGTAHRSHASNVGISEAMKAEGKQAAAGYLLEKEIKFLGNAVDSPKHPFVAILGGAKVSDKIGVINHLLGKADKVIVGGGMTYTFYAAKGIKIGNSLVEKDKIELAKEIMEKAGDKLVLPVDNVCAKEFNNDAPSEVVEGDIPDGYMALDIGPKSVELFEDTLKDAKTVVWNGPMGVFEMSNFAKGTLEVGKFLGTLSDATTIVGGGDSTAAVQQLGVADKLTHISTGGGASLEYLEGKTLPGIAAVSDK

SEQ ID NO: 4 - protein, 396 aa, Lactobacillus gasseri
  REGION 1..396: note = elongation factor tu
MAEKEHYERTKPHVNIGTIGHVDHGKTTLTAAITTVLAEDGLAQAEDYSQIDAAPEEKERGITINTAHVEYETKNRHYAHMDAPGHADYIKNMITGAAQMDGAILVVAATDGPMPQTREHILLARQVGVKYIVVFLNKVDLVDDPELIDLVEMEVRDLLTEYDYPGDDVPVIRGSALKALQGDPEQQDVIRKLMETVDEYIPTPERDTDKPFLMPVEDVFTITGRGTVASGRIDRGTVKVGDEVEIVGLTDKVEKSTVTGLEMFHKTLDLGEAGDNVGVLLRGIDRDQVERGQVLAAPGSIQTHKKFKGQVYILNKDEGGRHTPFFSDYRPQFYFHTTDVTGKIELPEGTEMVMPGDNVEFTVELIKPVAIEKGTKFTIREGGKTVGAGQVTEILD

SEQ ID NO: 5 - protein, 447 aa, Lactobacillus gasseri
  REGION 1..447: note = Glucose-6-phosphate isomerase
MSKVVHFDASKLTPFVHENELKEMQAMVTAADQELREGTGAGSDFRGWIDLPINYDKDEFDRIKKAAKKIQNDSEVLVGIGIGGSYLGAQASIEFLNSSFYGREKEKYPTVVFCGNSLSGSYLYDLLEWLGDKDFSINIISKSGTTTEPSIAFRVLKDKLIKKYGKEEAAKRIYATTDRAKGALKTEADAEGYEEFVVPDDIGGRFSVLSAVGLLPIAVAGGDIDAMMKGAADARAAYTDPDVSKDNPYQYAALRNILYRKGYTTELLENYEPSLRMFGEWWKQLMGESEGKDQKGIYPSSANFTTDLHSLGQYIQEGRRNLMETVIRVESPEHDVTIPDDKENLDQLNFLSGKTMNYVNDRAYEGVVLAHTDGGVPVMTVDIENQSAHTLGYLIYWFELAVGISGYLNGINPFNQPGVESYKRNMFGLLNKPGYEDLHDDLTKRLK

SEQ ID NO: 6 - protein, 403 aa, Lactobacillus gasseri
  REGION 1..403: note = Phosphoglycerate kinase
MAKLIVSDLDLKGKKVLVRVDFNVPIKNGVIGDDNRIVAALPTIKYIIEHGGKAILLSHLGRVKSDADKKELSLRPVAERLSELLKKPVTFVPENEGKEVEETIDKMKDGDVIVLENTRFQDIDNDFGKRESKNDPKLGEYWASLGDVFVNDAFGTAHRSHASNVGIATAMKKAGKPAAAGFLLEKEIKFLGDAVENPVHPFVTILGGAKVSDKIGVIENLIPKSDHILIGGGMAYTFLAAQGHKIGKSLFEADKVELAKELLAKAGDKIVLPVDNVAATEFSNDAPHEVVGDDIPDNEMGLDIGPKTVEKFRDILKDAKTVVWNGPMGAFEMPNYAEGTLEVGRALADLKDAVTIIGGGDSTAAAKQLGIAPKISHISTGGGASLNYLEGKELPGIACVSDK

SEQ ID NO: 7 - protein, 428 aa, Lactobacillus gasseri
  REGION 1..428: note = enolase1
MLKSVIENVHALEIFDSRGNPTVEVFVTLSNGVVGKAEVPSGASTGENEAVELRDGGSRLGGKGVMNAVNNVNTEINDALKGLDPHDQPNIDATMIALDGTPNKGRLGANAILGVSMATACAAAKDNHQPLYRYLGGTDLEMPQTFHNVINGGEHADNGIDIQEFMITPVAKTSFRDGFEKIVNVYHTLKKVLEDMGYETGLGDEGGFAPNMKNSEEALKALHESIIKAGYKPGEDIAIACDCAASYFYNKEDGKYHLEGKVLTDEELADYYDKLLDEFPELISMEDPYDENDVEGMVKFTESHKDRIQIVLDDFICTNPKLLNKAIHEGAGNASLIKLNQIGTVTETLETIRLSRKNGYNTMISHRSGETGDTFIADFAVAVNGGQLKSGAPARSERVEKYNRLLEIEEELGKGERLAFFPDNVDLD

SEQ ID NO: 8 - protein, 432 aa, Lactobacillus gasseri
  REGION 1..432: note = enolase2
MSVITDIHAREVLDSRGNPTVEAEVYTELGGFGRAIVPSGASTGEHEAVELRDGDKSRFGGQGVLTAVENVNGEIAKAVIGLDVTDQRLIDQTMIDLDGTPNKGRLGANAILSVSLASARAAADELGLPLYEYLGGPNAHVLPTPMMNVINGGKHADNNVDIQEFMIMPVGAKSLHEAVRMGAETFHTLKGLLQERGESTAVGDEGGFAPNLKNNEEPFEILVEAIQRAGYKPGQDIAIAFDCAASEFYNKDTKKYVTVADGREYTAEEWTSLIEDLVDKYPVISVEDPLDENDWEGWKTFTERLGDKVQIVGDDLFVTNTSYLEKGIKMGVANSILIKLNQIGTLTETFEAIEMAKEAGYTAVVSHRSGETEDTTIADLVVATNAGQIKTGSMSRTDRIAKYNQLMRIEEALGSTAQYKGIHSFYNLHKQF

SEQ ID NO: 9 - protein, 396 aa, Lactobacillus camelliae
  REGION 1..396: note = elongation factor tu
MAEKEHYERTKPHVNIGTIGHVDHGKTTLTAAITKVLAAKGLAKEQDYASIDAAPEEKERGITINTAHVEYETEKRHYAHIDAPGHADYVKNMITGAAQMDGAILVVAATDGPMPQTREHILLARQVGVDYIVVFLNKTDLVDDDELIDLVEMEVRELLSEYDFPGDDIPVIRGSALKALQGDPEQEKVIQELMDTVDEYIPTPVRDTDKPFLMPVEDVFTITGRGTVASGRIDRGTVKIGDEVEIVGLHDEVLKSVVTGLEMFRKTLDLGEAGDNVGILLRGINRDQVERGQVLAKPGSIQTHKKFKAEVYILTKEEGGRHTPFFSNYRPQFYFHTTDVTGVIELPDGVEMVMPGDNVTFEVDMIAPVALEKGTKFTVREGGRTVGAGVVSEILG

SEQ ID NO: 10 - protein, 286 aa, Lactobacillus camelliae
  REGION 1..286: note = Fructose tagatose bisphosphate aldolase
MFQAARKGHYAIGAFNTNNLEWTRALLKGAQELNVPILIQTSMGAAKYMGGYKLCKALIELEAESMNITVPVVIHLDHGNFEAAKEAIAAGYNSVMFDGHDLPFDENLEKTKEIVKLAHAKGISVEAEVGSIGGEEDGVIGEGELASVEEAKTLAATGIDFMAAGIGNIHGKYPSNWKGLHFDRLQEIADAVDLPLVLHGGSGIPQDQVEKAISMGIAKLNINTECQLAFAKATREYILANKDQEGKGYDPRKLLAPGTQAITDTFKEISGWMGTRPVKMVPEEIK

SEQ ID NO: 11 - protein, 290 aa, Lactobacillus camelliae
  REGION 1..290: note = 2,5-didehydrogluconate reductase
MAEISKMSDTYTLSNGVKIPIVGFGTWQTPDGDVAKHSVEVAIKDGYRHIDTAAMYRNEDSVGEGIKAGLAAAGLKRDDLFVTTKLNNNAHSFDLVQKAIDESLTKLGLDHVDLYLIHWPNPVSFRDSYPDSLKETWRGMEQVYKDGKARAIGISNFRQAHLDVIKADATVEPMVNQIFLNPSDLQPAVTKMNAENNILSEAYSPLGTGKLLGNEELAKVAAKYGKGVAQVLLRWSLQHGFLPLAKSVHDARIKENTELFDFTLSDEDMKTLDGMRGVAGLATDPDTTNF

SEQ ID NO: 12 - protein, 246 aa, Lactobacillus camelliae
  REGION 1..246: note = Triosephosphate isomerase
MAGNWKMNKNPEETQAFLDGVKGKLPESSKVQTVIAAPAIDLTTLVAGAKGTPLQTAAENCYFEDAGAFTGETSPKALKDMGLDYVVIGHSERRGYFHETDQDINKKAKAILKNGLLPIICCGESLDQREAGQTNDWVASQVEAALAGISAADVKNSVIAYEPIWAIGTGKTASADQAQEVVAHIRSIVAKLYDAETADAVRILYGGSVKPANVKELMAKPDIDGGLVGGASMDPESFVALANYQD

SEQ ID NO: 13 - protein, 230 aa, Lactobacillus camelliae
  REGION 1..230: note = 2,3-bisphosphoglycerate-dependent phosphoglycerate mutase
MAKLVLIRHGQSEWNLSNQFTGWVDVDLSEKGTEEAKEAGRRIKKAGLEFDQAYTSVLTRAIKTLHYALEESDQLWIPETKTWRLNERHYGALQGQNKAEAAKKWGDEQVHIWRRSYDTLPPLLSADDEGSAAKDRRYANLDPRIIPGGENLKVTLERVIPFWEDHIAPDLLDGKNVIIAAHGNSLRALTKYIENISDEDIINLEMTTGEPVVYTFNDKLEVTDKVKLSK

SEQ ID NO: 14 - protein, 395 aa, Lactobacillus plantarum [sequence omitted]

SEQ ID NO: 15 - protein, 518 aa, Lactobacillus plantarum
  REGION 1..518: note = GMP synthase
MAKTDTASFDSILVLDFGSQYNQLITRRIRDFGVYSELLPNTITAEEIKARHPKGIIFSGGPNSVYDDGAFRVDPEIFKLGLPILGICYGMQLMTYTLAGGKVESADNREYGKANIDVTSDSATLFKDTPAEQSVWMSHGDLVTQAPDGFDVVATSKNCPIAAIQDVDRKLYGIQFHAEVRNTDYGNDILRHFAFDVCQAEANWSMDDFIDMQIEKIRAEVGDKKVLLGLSGGVDSSVVGVLLHKAIGTQLTSIFVDHGLLRKGEADQVMASLEGKFGLNIIKVDAKDRFLSKLAGVSDPERKRKIIGNEFIQVFDEEATKLNGMEFLAQGTLYTDVIESGTSTAQTIKSHHNVGGLPEDMQFKLIEPLRTLFKDEARELGEKLGMPSDLVWRQPFPGPGLGIRVIGEITEDKLEIVRDSDFILRDEIKKAGLDREIWQYFTVLPGIKSVGVMGDGRTYDYTVGIRAVTSIDGMTADFARIPWDVLQKISVRIVNEVDHVNRIVYDVTSKPPSTIEWE

SEQ ID NO: 16 - protein, 605 aa, Lactobacillus plantarum
  REGION 1..605: note = Glutamine-fructose-6-phosphate aminotransferase
MCGIVGVTGKDSAVSILLNGLEKLEYRGYDSAGIYVNDQDGHDYLVKEKGRIDDLRKEVGEAVHGSTGIGHTRWATHGEPSVANAHPQVSADGRFYLVHNGVIENFEDLKQTYLSDVTFKSQTDTEVIVQLVDRFVTKEGLSTLAAFRKTLGLLGHSSYGFLLMDKEDPDTLYVAKNKSPLLIGVGEGFNVVCSDSLAMLDQTKDFLELHDGEIVVVKPDEIKITNQAGEPVERKPFHVDIDAAQADKGTYPFYMLKEIDEQPNVMRKLSQVYLNDAGDPIINDDLLTALKAADRLYIVAAGTSYHAGLVGAKLFESLANVPTEVHVSSEFAYNQPLLSAHPFFIFLTQSGETADSREVLLNVNDQHFPSLTITNVPNSTLSREATYTLLLHAGPEIAVASTKAYTAQIALQAILAKALGVAVDQPAATAFDVKQQLALVANGMQSLVDEKATFEKIAKSALLNTPNAFYIGRGLDYAVSLETALKLKEISYVQAEGFASGELKHGTIALIEKDTPVIGIITQKNTAGLTRSNLQEVAARGAKTVTIVTDSLAKDGDTVILPTVDERLTALLSVVPGQLLAYYTSLNKGLDVDKPRNLAKSVTVE

SEQ ID NO: 17 - protein, 396 aa, Lactobacillus reuteri
  REGION 1..396: note = Elongation factor Tu
MAEKEHYERTKPHVNIGTIGHVDHGKTTLTAAITKVLAAKGLAKAEDYADIDAAPEEKERGITINTAHVEYETEKRHYAHIDAPGHADYVKNMITGAAQMDGAILVVAATDGPMPQTREHILLARQVGVQYIVVFLNKTDLVDDDELVDLVEMEVRDLLSEYDFPGDDVPVVRGSALKALEGDPEQEKVILHLMDVIDDYIPTPKRPTDKPFMMPVEDVFTITGRGTVASGRIDRGTVKVGDEVEIVGLTEDVLKSTVTGLEMFHKTLDLGEAGDNVGVLLRGISHDQIQRGQVLAEPGSIQTHKNFKGEVYVMTKEEGGRHTPFFSNYRPQFYFHTTDVTGTIELPDGVEMVMPGDNVTFTVNLQKPVALEKGLKFTIREGGHTVGAGVVSDILD

SEQ ID NO: 18 - protein, 452 aa, Lactobacillus reuteri
  REGION 1..452: note = Glucose-6-phosphate isomerase
MTHIKFDSSALKQFVHENELGEMQAMVNAANDELRNGTGAGADFRDWLHLPTEYDKEEFARIKKAADKIQRDSDVLVVIGIGGSYLGAQMAIDFLHNTFYQAQNAKDRKAPLVVFAGNSLSSTYVHDLIQLIGDKDFSINVVSKSGTTTEPSIAFRIFKGLLIKKYGENEANKRIYATTDKTKGALKTEADAHGYETFVIPDGVGGRYSVLSAVGLLPIAASGADIDKLMEGAAQAEKDYVDPDLTKNEAYQYAAYRNILYRKGYETELLENYEPNMRMFAEWWKQLAGESEGKDQKGIYPSSANFTTDLHSLGQYIQEGRRFLMETVVKLDKPNYDMEIPTEPDNLDGLGYLEGKTMDYVNTKAYEAVVAAHTDGGVPVMTVHIPQEDEYTLGYLIYFFEVAMGISGYLNGINPFNQPGVEAYKTNMFGLLGKPGYEEIGKELRAKMDKND

SEQ ID NO: 19 - protein, 401 aa, Bifidobacterium dentium
  REGION 1..401: note = Phosphoglycerate kinase
MKTLKDLGDLKGKRVLVRADFNVPLDGTTITDDGRIKAALPTIKALREEGAKVILMAHLGRPKGKVVPELSLAPVATRLGELLGVEVPLASDTYGEDAQAKVAAMNDGDVVLLQNVRFNPEETSKDPEERAAYAKKIAALGEAFVSDGFGVVHRAQGSNYDVAADLPAAAGLLVEKEVKALSRATVDPERPLTVVLGGSKVSDKLGVIENLLDKADRLVIGGGMVYTFLKAKGLEVGTSLLEEDQVETVKGYIERAEKNGVELVLPTDIVVNPVFPKSDEDIAPEVVAADAIPADKMGLDIGPDSQKLFHDKIVDSKTVVWNGPMGVFEVPAFAAGTEAVAQGLVDATAAGAFTIVGGGDSASAVRNLGFPEDGFSHISTGGGASLEFLEGKELPGLKVLD

SEQ ID NO: 20 - protein, 825 aa, Bifidobacterium dentium
  REGION 1..825: note = phosphoketolase
MTSPVIGTPWKKLNAPVSEEAIEGVDKYWRAANYLSIGQIYLRSNPLMKEPFTREDVKHRLVGHWGTTPGLNFLIGHINRLIADHQQNTVIIMGPGHGGPAGTAQSYLDGTYTEYFPNITKDEAGLQKFFRQFSYPGGIPSHYAPETPGSIHEGGELGYALSHAYGAVMNNPSLFVPAIVGDGEAETGPLATGWQSNKLVNPRTDGIVLPILHLNGYKIANPTILSRISDEELHEFFHGMGYEPYEFVAGFDNEDHLSIHRRFAELFETVFDEICDIKAAAQTDDMTRPFYPMIIFRTPKGWTCPKFIDGKKTEGSWRSHQVPLASARDTEAHFEVLKNWLESYKPEELFDANGAVKPEVTAFMPTGELRIGENPNANGGRIREELNLPALEDYEVKEVAEYGHGWGQLEATRRLGVYTRDIIKNNPDSFRIFGPDETASNRLQAAYDVTNKQWDAGYLSAQVDEHMAVTGQVTEQLSEHQMEGFLEAYLLTGRHGIWSSYESFVHVIDSMLNQHAKWLEATVREIPWRKPISSMNLLVSSHVWRQDHNGFSHQDPGVTSVLLNKCFNNDHVIGIYFPVDSNMLLAVAEKCYKSTDMINAIIAGKQPAATWLTLDEARAELEKGAAEWEWASTAKSNDEAQIVLASAGDVPAQEIMAAADKLDAMGIKFKVVNVVDLVKLQSTKENDEAISDADFADLFTEDKPVLFAYHSYARDVRGLIYDRPNHDNFNVHGYEEQGSTTTPYDMVRVNNIDRYELVAEALRMIDADKYADKIDELEAFRKEAFQFAVDNGYDHPDYTDWVYSGVNTNKQGAVSATAATAGDNE

SEQ ID NO: 21 - protein, 399 aa, Bifidobacterium pseudolongum
  REGION 1..399: note = Elongation factor Tu
MAKEKYERTKPHVNIGTIGHVDHGKTTLTAAISKVLHDEFPDLNPEYDFNQIDAAPEEQQRGITINIAHIEYQTEKRHYAHVDCPGHADFVKNMITGAAQMDGAILVVAATDGPMAQTREHVLLARQVGVPKILVALNKCDMVDDEELIELVEEEVRDLLDENGFDRDCPVVHTSAYGALHDDAPDHEKWVETVKELMNDVDDYIPTPVHDLDKPFLMPIEDVFTISGRGTVVTGRVERGKLPINTNVEIVGIRPTQTTTVTSIETFHKQMDECEAGDNTGLLLRGLGRTDVERGQVVAAPGSVTPHTKFEGEVYVLTKDEGGRHSPFFSNYRPQFYFRTTDVTGVITLPEGVEMVQPGDHATFTVELIQPIAMEEGLTFAVREGGHTVGSGRVTKILA

SEQ ID NO: 22 - protein, 401 aa, Bifidobacterium pseudolongum
  REGION 1..401: note = phospoglycerate kinase
MKTLKDLGDLKGKRVLVRADFNVPLDGTTITDDGRIKAALPTIEELRKDGAKVILMAHLGRPKGKVVPELSLAPVAKRLSELLGCDVVLASDTYGPDAQTKVAAMNDGDVVLLENVRFNPEETSKDEAERAAYAKKIAALGDVFVSDGFGVVHRAQGSNYDVAADLPAAAGLLVEKEVKALSKATENPERPFTVVLGGSKVSDKLGVIENLLDKADRLVIGGGMVFTFLKAKGYEVGTSLLEEDQIDKVKGYIETAEKNGVELVLPVDIVVNKGFPAGDTPIDPQVVPADQIPSDMMGLDIGPESCKLFHDKIVDSKTVVWNGPMGVFEIPQFANGTRAVAQALVDATANGAFTIVGGGDSAAAVRNLGFPEDGFSHISTGGGASLEFLEGKELPGLKVLD

SEQ ID NO: 23 - protein, 401 aa, Bifidobacterium pseudolongum
  REGION 1..401: note = Phosphoglycerate kinase
MKTLKDLGDLKGKRVLVRADFNVPLDGTTITDDGRIKAALPTIEELRKDGAKVILMAHLGRPKGKVVPELSLAPVAKRLSELLGCDVVLASDTYGPDAQAKVAAMNDGDVVLLENVRFNPEETSKDEAERAEYAKKIAALGDVFVSDGFGVVHRAQGSNYDVAADLPAAAGLLVEKEVKALSKATENPERPFTVVLGGSKVSDKLGVIENLLDKADRLVIGGGMVFTFLKAKGYEVGTSLLEEDQIDKVKGYIETAEKNGVELVLPVDIVVNKGFPAGDTPIDPQVVPADQIPSDMMGLDIGPESCKLFHDKIVDSKTVVWNGPMGVFEIPQFANGTRAVAQALVDATANGAFTIVGGGDSAAAVRNLGFPEDGFSHISTGGGASLEFLEGKELPGLKVLD

SEQ ID NO: 24 - protein, 825 aa, Bifidobacterium pseudolongum
  REGION 1..825: note = Probable phosphoketolase
MTNPVIGTPWQKLDRPVSEEAIEGMDKYWRVTNYMSIGQIYLRSNPLMKEPFTRDDVKHRLVGHWGTTPGLNFLLAHINRLIADHQQNTVFIMGPGHGGPAGTSQSYVDGTYTEYYPNITKDEAGLQKFFRQFSYPGGIPSHFAPETPGSIHEGGELGYALSHAYGAVMNNPSLFVPCIIGDGEAETGPLATGWQSNKLVNPRTDGIVLPILHLNGYKIANPTILARISDEELHDFFRGMGYHPYEFVAGFDNEDHMSIHRRFAELFETIFDEICDIKAAAQTDDMTRPFYPMLIFRTPKGWTCPKFIDGKKTEGSWRAHQVPLASARDTEEHFEVLKGWLESYKPEELFNADGSIKDDVTAFMPKGDLRIGANPNANGGVIREDLKLPELDQYEVTGVKEYGHGWGQVEAPRSLGAYCRDIIKNNPDSFRIFGPDETASNRLNATYEVTDKQWDNGYLSSLVDEHMAVTGQVTEQLSEHQCEGFLEAYLLTGRHGIWSSYESFVHVIDSMLNQHAKWLEATVREIPWRKPISSVNLLVSSHVWRQDHNGFSHQDPGVTSVLINKTFNNDHVTNIYFATDANMLLAISEKCFKSTNKINAIFAGKQPAPTWITLDEARAELEAGAAEWKWASNAENNDEVQVVLAAAGDVPTQEIMAASDALNKMGIKFKVVNVVDLLKLQSRENNDEALTDEEFTDLFTADKPVLFAYHSYAQDVRGLIYDRPNHDNFNVVGYKEQGSTTTPFDMVRVNDMDRYALQAAALKMIDADKYADKIDELNAFRQKAFQFAVDNGYDIPEFTDWVYPDVKVDETQMLSATAATAGDNE

SEQ ID NO: 25 - protein, 825 aa, Bifidobacterium ruminantium
  REGION 1..825: note = Xylulose-5-phosphate/fructose-6-phosphate phosphoketolase
MTSPVIGTPWKKLNAPVSEEAIEGVDKYWRAANYLSIGQIYLRSNPLMKEPFTREDVKHRLVGHWGTTPGLNFLIGHINRLIADHQQNTVIIMGPGHGGPAGTAQSYLDGTYTEYFPNITKDEAGLQKFFRQFSYPGGIPSHYAPETPGSIHEGGELGYALSHAYGAVMNNPSLFVPAIVGDGEAETGPLATGWQSNKLINPRTDGIVLPILHLNGYKIANPTILSRISDEELHEFFHGMGYEPYEFVAGFDNEDHLSIHRRFAELFETVFDEICDIKAAAQTDDMTRPFYPMIIFRTPKGWTCPKFIDGKKTEGSWRSHQVPLASARDTEAHFEVLKNWLESYKPEELFDENGAVKPEVTAFMPTGELRIGENPNANGGRIREELKLPKLEDYEVKEVAEYGHGWGQLEATRRLGVYTRDIIKNNPDSFRIFGPDETASNRLQAAYDVTNKQWDAGYLSAQVDEHMAVTGQVTEQLSEHQMEGFLEGYLLTGRHGIWSSYESFVHVIDSMLNQHAKWLEATVREIPWRKPISSMNLLVSSHVWRQDHNGFSHQDPGVTSVLLNKCFNNDHVIGIYFPVDSNMLLAVAEKCYKSTNKINAIIAGKQPAATWLTLDEARAELEKGAAEWKWASNVKSNDEAQIVLAATGDVPTQEIMAAADKLDAMGIKFKVVNVVDLVKLQSAKENNEALSDEEFAELFTEDKPVLFAYHSYARDVRGLIYDRPNHDNFNVHGYEEQGSTTTPYDMVRVNNIDRYELQAEALRMIDADKYADKINELEAFRQEAFQFAVDKGYDHPDYTDWVYSGVNTNKQGAVSATAATAGDNE

SEQ ID NO: 26 - protein, 399 aa, Bifidobacterium adolescentis
  REGION 1..399: note = Elongation factor Tu
MAKEKYERTKPHVNIGTIGHVDHGKTTLTAAISKVLHEEYPDVNPEYDFNQIDSAPEEAARGITINIAHIEYQTEKRHYAHVDCPGHADFVKNMITGAAQMDGAILVVAATDGPMAQTREHVLLARQVGVPKILVALNKCDMVDDEELIELVEEEVRDLLDENGFDRDCPVIHVSAYGALHDDAPDHEKWVEQVKKLMDAVDDYIPTPVHDLDKPFLMPIEDVFTISGRGTVVTGRVERGKLPVNSNVEIVGIRPTQTTTVTSIETFHKQMDECEAGDNTGLLLRGINRDQVERGQVLAAPGSVTPHTKFEGEVYVLTKDEGGRHSPFFSNYRPQFYFRTTDVTGVITLPEGVEMVQPGDHATFGVELIQPIAMEEGLTFAVREGGHTVGSGRVTKIIE

SEQ ID NO: 27 - protein, 9 aa, Oryctolagus cuniculus
  REGION 1..9: note = oxytocin
CYIQNCPLG

SEQ ID NO: 28 - protein, 9 aa, synthetic construct
  REGION 1..9: note = 87-95 position fragment of SEQ ID NO:1
DYVKNMITG

SEQ ID NO: 29 - protein, 9 aa, synthetic construct
  REGION 1..9: note = 13-21 position fragment of SEQ ID NO:2
KFVNDNELG

SEQ ID NO: 30 - protein, 10 aa, synthetic construct
  REGION 1..10: note = 317-326 position fragment of SEQ ID NO:3
KTVVWNGPMG

SEQ ID NO: 31 - protein, 9 aa, synthetic construct
  REGION 1..9: note = 154-162 position fragment of SEQ ID NO:15
ATSKNCPIA

SEQ ID NO: 32 - protein, 9 aa, synthetic construct
  REGION 1..9: note = 371-379 position fragment of SEQ ID NO:16
LTITNVPNS

SEQ ID NO: 33 - protein, 7 aa, synthetic construct
  REGION 1..7: note = 313-319 position fragment of SEQ ID NO:5
QYIQEGR

SEQ ID NO: 34 - protein, 10 aa, synthetic construct
  REGION 1..10: note = 12-21 position fragment of SEQ ID NO:18
KQFVHENELG

SEQ ID NO: 35 - protein, 9 aa, synthetic construct
  REGION 1..9: note = 88-96 position fragment of SEQ ID NO:4
DYIKNMITG

SEQ ID NO: 36 - protein, 12 aa, synthetic construct
  REGION 1..12: note = 160-171 position fragment of SEQ ID NO:8
VDIQEFMIMPVG

SEQ ID NO: 37 - protein, 9 aa, synthetic construct
  REGION 1..9: note = 246-254 position fragment of SEQ ID NO:7
SYFYNKEDG

SEQ ID NO: 38 - protein, 8 aa, synthetic construct
  REGION 1..8: note = 1-8 position fragment of SEQ ID NO:10
MFQAARKG

SEQ ID NO: 39 - protein, 9 aa, synthetic construct
  REGION 1..9: note = 202-210 position fragment of SEQ ID NO:11
AYSPLGTGK

SEQ ID NO: 40 - protein, 8 aa, synthetic construct
  REGION 1..8: note = 61-68 position fragment of SEQ ID NO:12
CYFEDAGA

SEQ ID NO: 41 - protein, 9 aa, synthetic construct
  REGION 1..9: note = 191-199 position fragment of SEQ ID NO:13
KYIENISDE

SEQ ID NO: 42 - protein, 10 aa, synthetic construct
  REGION 1..10: note = 40-49 position fragment of SEQ ID NO:20
IYLRSNPLMK

SEQ ID NO: 43 - protein, 9 aa, synthetic construct
  REGION 1..9: note = 164-172 position fragment of SEQ ID NO:21
GFDRDCPVV

SEQ ID NO: 44 - protein, 9 aa, synthetic construct
  REGION 1..9: note = 89-97 position fragment of SEQ ID NO:21
DFVKNMITG

(19) United States
(12) Patent Application Publication
(10) Pub. No.: US 2025/0352589 A1
(43) Pub. Date: Nov. 20, 2025

(54) OXYTOCIN-MIMETIC COMPOSITIONS AND USES THEREOF

(71) Applicants: INSTITUT NATIONAL DE LA SANTÉ ET DE LA RECHERCHE MÉDICALE, Paris Cedex 13 (FR); UNIVERSITÉ DE ROUEN, Mont-Saint-Aignan (FR); TARGEDYS, Longjumeau (FR)

(72) Inventors: Serguei FETISSOV, Montigny (FR); Marion NICOL, Bois-Guillaume (FR); Clémentine PICOLO, Longjumeau (FR); Grégory LAMBERT, Longjumeau (FR)

(21) Appl. No.: 18/726,256

(22) PCT Filed: Sep. 16, 2022
(86) PCT No.: PCT/EP2022/075858

§ 371 Date: Jul. 2, 2024

(30) Foreign Application Priority Data
Nov. 3, 2021 (EP) 21306546.9

(51) Int. Cl. A61K 35/747; A61K 35/745; A61K 38/16; A61P 25/22; A61P 25/24

(52) U.S. Cl. CPC A61K 35/747; A61K 35/745; A61K 38/164; A61P 25/22; A61P 25/24

(57) ABSTRACT

The present invention relates to a composition comprising a probiotic composition comprising a Lactobacillus salivarius strain and a Lactobacillus gasseri strain. The invention further relates to the nutraceutical and therapeutical uses of such compositions in oxytocin-related neuropsychiatric disorders, in particular autism or anxiety related disorders selected from depression and eating disorders.

### SEQUENCE LISTING

This application includes as the Sequence Listing the complete contents of the xml file “15920005US_sub_seq_listing”, created Dec. 2, 2024, containing 67,960 bytes, hereby incorporated by reference.

### FIELD OF INVENTION

The present invention relates to a composition comprising a probiotic composition of a Lactobacillus salivarius strain and a Lactobacillus gasseri strain as well as its use in the treatment of oxytocin-related disorders.

### BACKGROUND OF INVENTION

According to the World Health Organization (2020), depression affects more than 264 million people worldwide. Many of those also suffer from anxiety. This does not only have a negative impact in the health of those suffering from them, but also on their life quality. Current research states that both disorders are triggered by the interaction of psychological, environmental, genetic, and biological factors. There are many therapeutic options to treat these disorders. However, these options often take a long time to work, cause mood swings, alterations in sleeping patterns, dependence and addiction, and health affections in other parts of the body.

Thus, there is a need to supply compositions for their use in treating neurocognitive dysfunctions such as depression and/or anxiety.

Recent scientific advances have shown a link between the gut microbiota and the host central nervous system, and the communication between them occurs via a bi-directional pathway termed the “microbiota-gut-brain axis.”. For instance, the international application WO2015/082633 discloses the “microbiota-gut-brain axis” cross-link between gut bacteria and the host nervous system via bacterial protein mimetics of neuropeptides. Indeed, caseinolytic protease B (ClpB) of Enterobacteriaceae has been identified as a conformational mimetic of alpha-melanocyte-stimulating hormone (alpha-MSH), a neuropeptide regulating feeding behavior and emotion in the context of eating disorders. Nevertheless, the general aspects of the links between the gut microbiota and depression have not been systematically investigated.

The Chinese application CN 111 821 321 discloses a probiotic composition for use the female reproductive system comprising a probiotic mixture of Lactobacillus salivarius, L. paracasei, L. acidophilus, L. reuteri, L. inert, L. gasseri, L. crispatus and L. johnsonii. CN 111 821 321 discusses the use of the disclosed composition in the discharging of inflammatory secretions and toxins of the female reproductive system and the improvement of emotional restlessness, irritability and depression caused by the menstruation, child birth or menopause.

However, there is a need to supply a composition for use in the emotional distress associated with the every-day life situations, such as anxiety and in particular chronic stress, beyond the human hormonal variations. The gut microbiome in the modern environment has markedly changed in response to natural and technological factors.

Indeed, the modern life-style may have an impact on a broad range of host physiology including mood namely in view of the every-day stress that humans are subjected to. Thus, there is still a need to supply compositions to addressing neuropsychiatric and emotional dysfunctions, in particular those associated with anxiety.

Oxytocin (OT) is a neuropeptide involved in a variety of physiological functions including key roles in stress, emotion, mood, social and feeding behavior and displays anti-stress, anxiolytic, pro-social, analgesic and anti-depressive effects. Furthermore, the findings of Penagarikano et al. have added exciting support for using oxytocin in autistic spectrum disorders (Penagarikano et al. Sci. Transl. Med. 2015; 7:271ra8). The inventors, surprisingly found bacterial strains that act on the microbiota-gut-brain axis and thus neurocognitive functions via the oxytocin pathway. Thus, the present invention supplies novel compositions for use in the treatment of oxytocin pathway-associated dysfunctions, particularly autism or anxiety related disorders selected from depression and eating disorders such as sugar craving or binge eating.

The present invention further addresses this problem by supplying nutraceutical and pharmaceutical uses of the compositions detailed hereinbelow to ensure the well-being of a subject.

### SUMMARY

In a first aspect, the invention relates to a composition for use in the treatment of autism or anxiety related disorders selected from depression and eating disorders; wherein the composition comprises a probiotic composition comprising a Lactobacillus salivarius strain and a Lactobacillus gasseri strain.

It was found that the proteins expressed by the probiotic composition present an oxytocin-like immunoreactivity and/or directly stimulate the oxytocin receptor. Thus, the use according to the invention may be mediated by oxytocin-like immunoreactivity and/or by stimulation of the oxytocin receptor.

Indeed, it was found that the probiotic composition expresses a bioactive peptide mixture comprising at least one, preferably at least four, even more preferably at least five bioactive peptides selected from the group consisting of presenting from at least 80%, from at least 85%, preferably from at least 90%, even more preferably from at least 95%, or 100% identity with any one the peptides of SEQ ID 28, SEQ ID NO 29, SEQ ID NO 30, SEQ ID NO 33, SEQ ID NO 35, SEQ ID NO 36, and SEQ ID NO 37.

The anxiety-related disorders may also be referred to as chronic mild stress related disorders. Thus, in one embodiment, the anxiety according to the invention is chronic mild stress.

Typically, the anxiety related eating disorders are selected from binge eating and sugar craving.

According to some embodiments, the probiotic composition consists of a Lactobacillus salivarius strain and a Lactobacillus gasseri strain.

In one embodiment, the Lactobacillus salivarius strain is Lactobacillus salivarius DSM 16530 strain.

The invention also refers to a composition comprising a probiotic composition consisting of a Lactobacillus salivarius strain and a Lactobacillus gasseri strain.

The Lactobacillus salivarius strain of the composition may be Lactobacillus salivarius DSM 16530 strain and/or the Lactobacillus gasseri strain may be Lactobacillus gasseri LMG P-29638 strain.

Besides to probiotic composition, the composition of the invention may further comprise:
at least one plant or plant extract, preferably selected from the group consisting of Rhodiola rosea, Withania somnifera, Melissa officinalis, Crocus sativus and Hypericum perforatum;
at least one magnesium supplement preferably selected from magnesium bound to an ammo acid or a magnesium salt selected from the group consisting of magnesium carbonate, magnesium oxide, magnesium acetate, magnesium ascorbate, magnesium citrate, magnesium gluconate, magnesium lactate, magnesium malate, magnesium pyrrolidone carboxylate, magnesium taurate, and magnesium threonate; and/or
at least one micronutrient selected from calcium, magnesium, phosphorus, iron, zinc, copper, iodine, selenium, beta carotene, Vitamin C, Vitamin B1 Vitamin B6, Vitamin B2, niacin, Vitamin B12, folic acid, biotin, Vitamin D, and Vitamin E.

The composition according to the invention may be formulated in an oral dosage form selected from enterically-coated tablets and enterically-coated capsules, wherein the enteric-coating is a mixture comprising hydroxypropyl methyl cellulose and gellan gum.

Furthermore, the composition according to the invention may be formulated in a moisture-tight blister or container.

Lastly, the invention relates a composition comprising a mixture of bioactive peptides comprising or consisting of at least one, preferably at least four, bioactive peptides, wherein the at least one bioactive peptide is selected from the group consisting of peptides presenting from at least 80%, from at least 85%, preferably from at least 90%, even more preferably from at least 95%, or 100% identity with any one the peptides of SEQ ID 28, SEQ ID NO 29, SEQ ID NO 30, SEQ ID NO 33, SEQ ID NO 35, SEQ ID NO 36, and SEQ ID NO 37, for use in the treatment of autism or anxiety related disorders selected from depression and eating disorders.

### Definitions

In the present invention, the following terms have the following meanings:

“About” preceding a figure means plus or less 10% of the value of said figure, preferably plus or less 5% of the value of said figure, in particular plus or less 1% of the value of said figure.

“Excipients” refers to any inactive ingredient which is required for the formulation of an active agent in a suitable dosage form. Excipients are materials suitable for administration and include any such material known in the art which is non-toxic and which does not interact with any components of the composition in a deleterious manner.

In one embodiment, “Excipients” refers to any and all solvents, diluents carriers, fillers, bulking agents, binders, disintegrants, polymer, lubricant, glidant, surfactants, isotonic agents, thickening or emulsifying agents, stabilizers, absorption accelerators, flavoring agents, preservatives, antioxidants, buffering agents, or any combination thereof.

“Food” refers to liquid (i.e. drink), solid or semi-solid dietetic compositions, especially total food compositions (food-replacement), which do not require additional nutrient intake or food supplement compositions. Food supplement compositions do not completely replace nutrient intake by other means. Food and food supplement compositions are for example fermented dairy products or dairy-based products, which are preferably administered or ingested orally one or more times daily. Fermented dairy products can be made directly using the bacteria according to the invention in the production process, e.g. by addition to the food base, using methods known per se. In such methods, the strain(s) of the invention may be used in addition to the micro-organism usually used, and/or may replace one or more or part of the micro-organism usually used. For example, in the preparation of fermented dairy products such as yoghurt or yoghurt-based drinks, a combination of bacteria of the invention may be added to or used as part of a starter culture or may be suitably added during such a fermentation. Optionally, the bacteria may be inactivated or killed later in the production process. Fermented dairy products include milk-based products, such as (but not limited to) deserts, yoghurt, yoghurt drinks, quark, kefir, fermented milk-based drinks, buttermilk, cheeses, dressings, low fat spreads, fresh cheese, soy-based drinks, ice cream, etc. Alternatively, food and/or food supplement compositions may be non-dairy or dairy non fermented products (e.g. strains or cell-free medium in non-fermented milk or in another food medium). In some embodiments, the strains used in the present invention are encapsulated and dispersed in a food (e.g. in milk) or non-food medium. Non-fermented dairy products may include ice cream, nutritional bars and dressings, and the like. Non-dairy products may include powdered beverages and nutritional bars, and the like. The products may be made using known methods, such as adding an effective amount of the strain(s) and/or cell-free culture medium to a food base, such as skimmed milk or milk or a milk-based composition and fermentation as known. Other food bases to which the (compositions comprising the) bacterial cells and/or cell-free culture medium may be added are meat, meat replacers or plant bases.

“Food ingredient” or “feed ingredient” refers to a formulation which is or can be added to functional foods or foodstuffs as a nutritional supplement.

“Nutritional food” or “nutraceutical” or “functional” food refers to a foodstuff which contains ingredients having beneficial effects for health or capable of improving physiological functions.

“Food supplement” refers to a foodstuff having the purpose of completing normal food diet. A food supplement is a concentrated source of nutrients or other substances having a nutritional or physiological effect, when they are taken alone or as a combination in small amounts.

“Pharmaceutical composition” refers to a composition comprising an active principle in association with a pharmaceutically acceptable vehicle or excipient. A pharmaceutical composition is for therapeutic use, and relates to health. Especially, a pharmaceutical composition may be indicated for treating a disease or disorder.

By “pharmaceutically acceptable” is meant that the ingredients of a pharmaceutical composition are compatible with each other and not deleterious to the subject to which it is administered.

“Pharmaceutically acceptable excipient” refers to an excipient that does not produce an adverse, allergic or other untoward reaction when administered to an animal, preferably a human. It includes any and all solvents, dispersion media, coatings, antibacterial and antifungal agents, isotonic and absorption delaying agents and the like. For human administration, preparations should meet sterility, pyrogenicity, general safety and purity standards as required by regulatory offices, such as, for example, FDA Office or EMA. The terms “pharmaceutically acceptable excipient”, “pharmaceutically acceptable carrier” or “pharmaceutical vehicle” refer to an inert medium or carrier used as a solvent or diluent in which the pharmaceutically active ingredient is formulated and/or administered, and which does not produce an adverse, allergic or other reaction when administered to an animal, preferably a human being. This includes all solvents, dispersion media, coatings, antibacterial and antifungal agents, isotonic agents, absorption retardants and other similar ingredients. For human administration, preparations must meet standards of sterility, general safety and purity as required by regulatory agencies such as the FDA or EMA. For the purposes of the invention, “pharmaceutically acceptable excipient” includes all pharmaceutically acceptable excipients as well as all pharmaceutically acceptable carriers, diluents, and/or adjuvants.

“Pharmaceutically effective amount” refers to the amount of a pharmaceutical composition necessary and sufficient for slowing down or stopping the progression, aggravation, or deterioration of one or more symptoms of a disease, disorder or condition; or alleviating the symptoms of a disease, disorder or condition; or curing the disease, disorder or condition.

“Nutraceutical composition” refers to a composition comprising an edible ingredient allegedly providing a physiological benefit. A nutraceutical composition is for non-therapeutic use, and relates to comfort. The term “comfort” refers to the feeling of ease or well-being. Especially, a nutraceutical composition may be used for promoting, maintaining and/or improving comfort or for alleviating and/or preventing a discomfort. A nutraceutical composition may be in the form of a nutritional product, such as, for example, a functional food or a food or dietary supplement.

“Nutraceutically effective amount” refers to the amount of a nutraceutical composition, food or dietary supplement or functional food necessary and sufficient for providing a physiological benefit or alleviating a discomfort.

The term “administration”, or a variant thereof (e.g., “administering”), means providing the active agent, herein the strain combination, alone or as part of a pharmaceutically acceptable composition, to the patient in whom/which the condition, symptom, or disease is to be treated.

The term “subject” refers to a mammal, preferably a human. According to the present invention, a subject is a mammal, preferably a human. In one embodiment, the subject is a “patient”, i.e., a mammal, preferably a human, who/which is awaiting the receipt of, or is receiving medical care or was/is/will be the object of a medical procedure or is monitored for the development of a condition, symptom, disorder or disease.

The term “human” refers to a subject of both genders and at any stage of development (i.e., neonate, infant, juvenile, adolescent, adult). In one embodiment, the subject is not a female subject, nor a female human that undergoes menstruation, menopause or under post-partum conditions.

The term “therapeutically effective amount” (or more simply an “effective amount”) refers to the amount of active agent, herein the strain combination, alone or as part of a pharmaceutical composition, that is aimed at, without causing significant negative or adverse side effects to the subject in need of treatment, preventing, reducing, alleviating or slowing down (lessening) one or more of the symptoms of a condition, symptom, disorder or disease.

The terms “treat”, “treating” or “treatment”, as used herein, refer to a therapeutic treatment, to a prophylactic (or preventive) treatment, or to both a therapeutic treatment and a prophylactic (or preventive) treatment, wherein the object is to prevent, reduce, alleviate, and/or slow down (lessen) one or more of the symptoms of a condition, symptom, disorder or disease, in a subject in need thereof.

### DETAILED DESCRIPTION

### Strains' Compositions

The present invention refers to compositions comprising a probiotic composition of bacteria which produce oxytocin-like immunoreactive proteins.

The Applicants have found that the combination of Lactobacillus salivarius and Lactobacillus gasseri strains presents several peptides with an oxytocin mimetic activity among which the peptides and fragments showing a pronounced effect on the oxytocin-receptor, thereby rendering possible to treat oxytocin-pathway mediated diseases such as autism or anxiety related disorders. Furthermore, in vivo evidence showed a surprising effect of the combination of L. salivarius and L. gasseri strains on anxiety related disorders such as anxiety-related, in particular chronic stress-related depression and eating disorders.

Thus, the present invention relates to a composition comprising a probiotic composition comprising a Lactobacillus salivarius strain and a Lactobacillus gasseri strain. It is understood that the composition of the invention may further comprise non-probiotic constituents, such as for example pharmaceutical/nutraceutical excipients, plants or plant extracts or vitamins as indicatively hereinafter described. In other terms, the composition may comprise a probiotic fraction and a non-probiotic fraction.

According to one embodiment, the probiotic composition (or fraction) consists of a Lactobacillus salivarius strain and a Lactobacillus gasseri strain. Such embodiment may be of interest with regard to supplying an effective composition according to the invention in order to limit the competition among probiotic strains in the colonization of the treated subject. Furthermore, such embodiment may be of interest in the purview of supplying a simple yet efficient composition for the uses described herein.

“Mimetic” refers to a protein that imitates another protein. This imitation is possible since the protein shares certain characteristics with the protein it mimics.

In one embodiment, oxytocin-like or oxytocin-mimetic activity refers to immune conformational mimetism wherein the at least one protein cross-reacts with anti-oxytocin antibodies. In one embodiment, oxytocin-like activity refers to the oxytocin receptor agonism wherein the at least one protein, typically a fraction thereof, activates the oxytocin receptor. In one embodiment, oxytocin-like activity refers to immune conformational mimetism and/or oxytocin receptor agonism. A “conformational mimetic” refers to a protein, that shares at least in part the same conformation as another protein.

Lactobacillus salivarius is a Gram-positive non-sporulating bacterial species. It is a homofermentative organism that produces only a by-product of metabolism, lactic acid, and is naturally found in the human oral cavity, intestines and vaginal mucosa. It is considered non-pathogenic, and is sometimes used to produce lactic acid in fermented foods, probiotics, to help prevent infections with other microorganisms. In humans, L. salivarius has been used to prevent and treat a variety of diseases such as asthma, cancer, atopic dermatitis and halitosis, and to a limited extent, to prevent or treat infections (Chaves et al., J. Appl. Microbiol. 2017; 123(1), pp. 18-28). According to the scientific review by Chaves et al., it seems that the use, in particular the oral use such as the oral probiotic use, of L. salivarius does not pose a health risk to animals or humans in the doses currently used for a variety of applications.

The inventors found out that L. salivarius expresses the following proteins that surprisingly immune cross-react with anti-oxytocin antibodies and thus present an oxytocin-like effect:
Elongation factor Tu having an amino acid sequence of SEQ ID NO:1,
Glucose-6-phosphate isomerase having an amino acid sequence of SEQ ID NO:2, and
Phosphoglycerate kinase having an amino acid sequence of SEQ ID NO:3.

In one specific embodiment, L. salivarius is the L. salivarius DSM 16530 strain such as for example the L. salivarius LS7892 produced by CSL/Sacco.

Thus, in one embodiment, the probiotic composition comprises or consists of a L. salivarius strain and a L. gasseri strain, wherein the L. salivarius strain is the L. salivarius DSM 16530 strain.

Lactobacillus gasseri is a lactic acid bacterium from the Lactobacillaceae family. It is part of the microbiota of the oral, vaginal and intestinal mucous membranes of humans. Just like L. salivarius, L. gasseri is a well-known bacterial strain for probiotic applications.

The inventors found out that L. gasseri expresses the following proteins that surprisingly immune cross-react with anti-oxytocin antibodies and thus present an oxytocin-like effect:
Elongation factor Tu having an amino acid sequence of SEQ ID NO:4,
Glucose-6-phosphate isomerase having an amino acid sequence of SEQ ID NO:5,
Phosphoglycerate kinase having an amino acid sequence of SEQ ID NO:6,
Enolase 1 having an amino acid sequence of SEQ ID NO:7, and
Enolase 2 having an amino acid sequence of SEQ ID NO:8.

In one specific embodiment, L. gasseri is the L. gasseri LMG P-29638 strain, such as for example L. gasseri LG6410 produced by CSL/Sacco.

Thus, in one embodiment, the probiotic composition comprises or consists of a L. salivarius strain and L. gasseri strain, wherein the L. gasseri strain is the L. gasseri LMG P-29638 strain.

Considering the Oxytocin-mimetic profile of each of the probiotic strains according to the invention, the probiotic composition comprising L. salivarius and L. gasseri inherently expresses proteins that immune cross-react with anti-oxytocin antibodies and thus present an oxytocin-like effect, said proteins being selected from SEQ ID NO:1 to SEQ ID NO:8, thereby presenting an oxytocin-like immunoreactivity.

The presence of oxytocin-like motifs in the above target proteins was searched by sequence alignment with the oxytocin amino acid sequence (SEQ ID NO: 27). Methods for comparing the identity of two or more sequences are well known in the art. The “needle” program, which uses the Needleman-Wunsch global alignment algorithm (Needleman and Wunsch, 1970 J. Mol. Biol. 48:443-453) to find the optimum alignment (including gaps) of two sequences when considering their entire length, may for example be used. The needle program is, for example, available on the ebi.ac.uk world wide web site. The percentage of identity in accordance with the invention is preferably calculated using the EMBOSS: needle (global) program with a “Gap Open” parameter equal to 10.0, a “Gap Extend” parameter equal to 0.5, and a Blosum62 matrix.

It was surprisingly found that the following fragments of proteins expressed by the L. salivarius and/or L. gasseri strains not only present an homology with the oxytocin amino acid sequence (SEQ ID NO: 27) but also can strongly activate the oxytocin receptor at a micromolar scale:
SEQ ID 28: H—DYVKNMITG—OH being the 87-95 position fragment of SEQ ID NO:1, typically expressed by L. salivarius,
SEQ ID NO 29: H—KFVNDNELG—OH being the 13-21 position fragment of SEQ ID NO:2, typically expressed by L. salivarius,
SEQ ID NO 30: H—KTVVWNGPMG—OH being the 317-326 position fragment of SEQ ID NO:3, typically expressed by L. salivarius and L. gasseri,
SEQ ID NO 33: H—QYIQEGR-OH being the 313-319 position fragment of SEQ ID NO:5, typically expressed by L. gasseri,
SEQ ID NO 35: H—DYIKNMITG—OH being the 88-96 position fragment of SEQ ID NO:4, typically expressed by L. gasseri,
SEQ ID NO 36: H—VDIQEFMIMPVG—OH being the 160-171 position fragment of SEQ ID NO:8, typically expressed by L. gasseri,
SEQ ID NO 37: H—SYFYNKEDG—OH being the 246-254 position fragment of SEQ ID NO:7, typically expressed by L. gasseri,

Thus, the probiotic composition expresses a bioactive peptide mixture comprising at least one, preferably at least four, even more preferably at least five bioactive peptides selected from the group consisting of peptides presenting from at least 80%, from at least 85%, preferably from at least 90%, even more preferably from at least 95%, or 100% identity with any one the peptides of SEQ ID 28, SEQ ID NO 29, SEQ ID NO 30, SEQ ID NO 33, SEQ ID NO 35, SEQ ID NO 36, and SEQ ID NO 37, thereby being capable of simulating the oxytocin receptor.

The amount of each strain in the composition of the invention may vary in a wide range. The amount of each strain in the composition of the invention may be comprised between 10² and 10¹³ CFU, preferably between 10⁵ and 10¹² CFU, in particular between 10⁹ and 10¹⁰ CFU. In some embodiments, the amount of each strain in the composition is about 10², 10³, 10⁴, 10⁵, 10⁶, 10⁷, 108, 10⁹, 10¹⁰, 10¹¹, 10¹² or 10¹³ CFU. One of ordinary skill in the art is able to determine, depending on the strains, on the other components of the composition, and on the desired effect of the composition, the appropriate amount of each strain in the composition.

The total amount of strains in the composition of the invention may vary in a wide range. The total amount of strains in the composition of the invention may be comprised between 10⁴ and 10¹⁵ CFU, preferably between 10⁶ and 10¹³ CFU, in particular between 10⁹ and 10¹¹ CFU. In some embodiments, the amount of each strain in the composition is about 10⁴, 10⁵, 10⁶, 10⁷, 10⁸, 10⁹, 10¹⁰, 10¹¹, 10¹², 10¹³, 10¹⁴ or 10¹¹ CFU. One of ordinary skill in the art is able to determine, depending on the strains, on the other components of the composition, and on the desired effect of the composition, the appropriate total amount of strains in the composition.

The relative amount of one strain to the other strain(s) in the composition may vary in a wide range, depending, among others, on the number of different strains in the composition. In some embodiments, each strain is present in an equal amount. In such an embodiment, if the composition of the invention comprises exactly two strains, each strain is present in an amount that is half the total amount of strains. In another such embodiment, if the composition of the invention comprises exactly three strains, each strain is present in an amount that is a third of the total amount of strains. In other embodiments, the amount of the different strains of the composition is not the same.

In one exemplary embodiment, the probiotic composition comprises, preferably consists of 10¹⁰ CFU of Lactobacillus salivarius and 10¹⁰ CFU of Lactobacillus gasseri.

Thus, in one embodiment the composition comprises a probiotic composition comprising, preferably consisting of a L. salivarius strain and a L. gasseri strain, preferably in an amount ranging from 10⁶ and 10¹³ CFU each, even more preferably the L. salivarius strain being the L. salivarius DSM 16530 strain and optionally the L. gasseri strain being the L. gasseri LMG P-29638 strain;

The composition of the invention may further comprise any suitable other components known in the art.

In an embodiment, the composition according to the invention further comprises at least one plant or plant extract, preferably selected from the group consisting of Rhodiola rosea, Withania somnifera, Melissa officinalis, Crocus sativus and Hypericum perforatum, even more preferably the at least one plant or plant extract being Rhodiola rosea.

Thus, in one embodiment the composition comprises:
A probiotic composition comprising, preferably consisting of a L. salivarius strain and a L. gasseri strain, preferably in an amount ranging from 10⁶ and 10¹³ CFU each, even more prefererably the L. salivarius strain being the L. salivarius DSM 16530 strain and optionally the L. gasseri strain being the L. gasseri LMG P-29638 strain; and
a plant or plant extract, preferably selected from the group consisting of Rhodiola rosea, Withania somnifera, Melissa officinalis, Crocus sativus and Hypericum perforatum, even more preferably the at least one plant or plant extract being Rhodiola rosea.

In an embodiment, the composition according to the invention further comprises at least one mineral supplement, preferably a magnesium supplement. The magnesium supplement may be selected from a magnesium salt or magnesium bound to an ammo acid. In one embodiment, the magnesium salt is selected from the group consisting of magnesium carbonate, magnesium oxide, magnesium acetate, magnesium ascorbate, magnesium citrate, magnesium gluconate, magnesium lactate, magnesium malate, magnesium pyrrolidone carboxylate, magnesium taurate, and magnesium threonate.

Thus, in one embodiment the composition comprises:
A probiotic composition comprising, preferably consisting of a L. salivarius strain and a L. gasseri strain, preferably in an amount ranging from 10⁶ and 10¹³ CFU each, even more preferably the L. salivarius strain being the L. salivarius DSM 16530 strain and optionally the L. gasseri strain being the L. gasseri LMG P-29638 strain; and
a magnesium supplement preferably selected from a magnesium salt or magnesium bound to an ammo acid, even more preferably the magnesium supplement being a magnesium salt selected from magnesium carbonate, magnesium oxide, magnesium acetate, magnesium ascorbate, magnesium citrate, magnesium gluconate, magnesium lactate, magnesium malate, magnesium pyrrolidone carboxylate, magnesium taurate, and magnesium threonate.

The composition may further comprise, at least one micronutrient selected from calcium, magnesium, phosphorus, iron, zinc, copper, iodine, selenium, beta carotene, Vitamin C, Vitamin B1 Vitamin B6, Vitamin B2, niacin, Vitamin B12, folic acid, biotin, Vitamin D, and/or Vitamin E.

Thus, in one embodiment the composition comprises:
A probiotic composition comprising, preferably consisting of a L. salivarius strain and a L. gasseri strain, preferably in an amount ranging from 10⁶ and 10¹³ CFU each, even more preferably the L. salivarius strain being the L. salivarius DSM 16530 strain and optionally the L. gasseri strain being the L. gasseri LMG P-29638 strain; and
at least one micronutrient selected from calcium, magnesium, phosphorus, iron, zinc, copper, iodine, selenium, beta carotene, Vitamin C, Vitamin B1 Vitamin B6, Vitamin B2, niacin, Vitamin B12, folic acid, biotin, Vitamin D, and/or Vitamin E.

In one embodiment the composition comprises:
A probiotic composition comprising, preferably consisting of a L. salivarius strain and a L. gasseri strain, preferably in an amount ranging from 10⁶ and 10¹³ CFU each, even more preferably the L. salivarius strain being the L. salivarius DSM 16530 strain and optionally the L. gasseri strain being the L. gasseri LMG P-29638 strain;
and at least one of the following:
a plant or plant extract, preferably selected from the group consisting of Rhodiola rosea, Withania somnifera, Melissa officinalis, Crocus sativus and Hypericum perforatum, even more preferably the at least one plant or plant extract being Rhodiola rosea, and/or
a magnesium supplement preferably selected from a magnesium salt or magnesium bound to an ammo acid, even more preferably the magnesium supplement being a magnesium salt selected from magnesium carbonate, magnesium oxide, magnesium acetate, magnesium ascorbate, magnesium citrate, magnesium gluconate, magnesium lactate, magnesium malate, magnesium pyrrolidone carboxylate, magnesium taurate, and magnesium threonate, and/or
at least one micronutrient selected from calcium, magnesium, phosphorus, iron, zinc, copper, iodine, selenium, beta carotene, Vitamin C, Vitamin B1 Vitamin B6, Vitamin B2, niacin, Vitamin B12, folic acid, biotin, Vitamin D, and/or Vitamin E.

In some embodiments, the composition of the invention is a pharmaceutical composition and/or a nutraceutical composition.

Pharmaceutical and nutraceutical compositions according to the invention differ mainly in their intended use. Main features that may vary between pharmaceutical and nutraceutical compositions according to the invention are: the used strains combination, the form of the composition, the excipients and/or the dosing of each strain in the composition.

The composition of the invention may further comprise at least one excipient, preferably at least one pharmaceutically and/or nutraceutically acceptable excipient.

The composition of the invention, in particular the pharmaceutical and/or nutraceutical composition of the invention, may be formulated to be suitable for any type of administration. For instance, the composition of the invention, in particular the therapeutic and/or nutraceutical composition of the invention, may be formulated to be suitable for oral, rectal, percutaneous, permucosal and/or intravenous administration, and/or for administration by injection.

One skilled in the art is able to select the appropriate excipients and/or vehicles for preparing, from the strains, a composition that is suitable for any type of administration. A composition may thus be presented in the form of injectable solutions or suspensions or multi-dose bottles, in the form of plain or coated tablets, sugarcoated tablets, wafer capsules, gel capsules, pills, cachets, powders, suppositories or rectal capsules, solutions or suspensions such as for example solutions or suspensions for intranasal administration, for percutaneous use in a polar solvent, or for permucous use.

Examples of excipients that are suitable for such administrations are cellulose derivatives, microcrystalline cellulose derivatives, alkaline-earth metal carbonates, magnesium phosphate, starches, modified starches and lactose for the solid forms.

For rectal use, cocoa butter or polyethylene glycol stearates are the preferred excipients.

For parenteral and/or intranasal use, water, aqueous solutions, physiological saline and isotonic solutions are the vehicles that are the most suitable.

In some embodiments, the composition of the invention, in particular the therapeutic and/or nutraceutical composition of the invention, is an oral composition, in particular an oral therapeutic and/or nutraceutical composition. An oral composition is a composition that is suitable for oral administration. Suitable forms for oral administration include uncoated or coated tablets, sugar-coated tablets, capsules, gelatin capsules, pills, cachets, powders.

Suitable excipients for formulation in the form of a tablet or capsule include oral, non-toxic, pharmaceutically acceptable inert carriers such as ethanol, glycerol, water, and the like. Suitable binders, lubricants, disintegrants, and color developers may also be included in the mixture, if desired or desired. Suitable binders include, but are not limited to, natural sugars such as starch, gelatin, glucose or beta-lactose, corn sweeteners, natural and synthetic gums such as acacia, track can or sodium oleate, sodium stearate, magnesium stearate, sodium benzoate, sodium acetate, sodium chloride, and the like. Disintegrants include, but are not limited to, starch, methylcellulose, agar, bentonite, xanthan gum, and the like. Pharmaceutical carriers acceptable for compositions formulated as liquid solutions include, but are not limited to, saline, sterile water, Ringer's solution, buffered saline, albumin injection solutions, Dextrose solution, maltodextrin solution, glycerol, ethanol, and one or more of these components may be mixed and used, and other conventional additives such as antioxidants, buffers, bacteriostats, and the like may be added as necessary. Diluents, dispersants, surfactants, binders and lubricants may also be added to formulate into injectable formulations, pills, capsules, granules or tablets such as aqueous solutions, suspensions, emulsions and the like.

In some embodiments, the composition according to the invention is a nutraceutical composition. The nutraceutical composition according to the invention may be in particular a food composition.

The food composition according to the present invention can be added to various foods. Foods to which the composition of the present invention can be added include, for example, beverages, vitamin complexes, dietary supplements, and the like.

The food compositions of the present invention may comprise ingredients conventionally added in food preparation and include, for example, proteins, carbohydrates, fats, nutrients, flavoring agents and flavoring agents. Examples of the carbohydrates described above are monosaccharides such as glucose, fructose, and the like; disaccharides such as maltose, sucrose, oligosaccharide, and the like; and polysaccharides such as conventional sugars such as dextrin, cyclodextrin, and the like, and sugar alcohols such as xylitol, sorbitol, erythritol, and the like. Natural flavors (taumatin, stevia extracts (e.g., rebaudioside a, glycyrrhizin, etc.)) and synthetic flavors (saccharin, aspartame, etc.) can be used as flavors. For example, when the food composition of the present invention is prepared from a drink and a beverage, citric acid, liquid fructose, sugar, glucose, acetic acid, malic acid, fruit juice, and various plant extracts may be further included.

In some embodiments of the composition according to the invention, the strains are encapsulated in order to be protected against the stomach. Accordingly, in some embodiments, the strains are in an encapsulated form so as significantly to improve their survival time. In such a case, the presence of a capsule may in particular delay or prevent the degradation of the microorganisms in the gastrointestinal tract. It will be appreciated that the compositions of the present embodiments can be encapsulated into an enterically-coated, time-released capsule or tablet. The enteric coating allows the capsule/tablet to remain intact (i.e., undissolved) as it passes through the gastrointestinal tract, until such time as it reaches the small intestine. Methods of encapsulating live bacterial cells are well known in the art (see, e.g., U.S. patents to General Mills Inc. such as U.S. Pat. No. 6,723,358). For example, micro-encapsulation with alginate and Hi-Maize™ starch followed by freeze-drying has been proved successful in prolonging shelf-life of bacterial cells in dairy products [see, e.g., Kailasapathy et al. Curr Issues Intest Microbiol. 2002 September; 3(2):39-48]. Alternatively, encapsulation can be done with glucomannane fibers such as those extracted from Amorphophallus konjac. Alternatively, entrapment of viable probiotic in sesame oil emulsions may also be used [see, e.g., Hou et al. J. Dairy Sci. 86:424-428]. In some embodiments, agents for enteric coatings are preferably methacrylic acid-alkyl acrylate copolymers, such as Eudragit® polymers. Poly(meth)acrylates have proven particularly suitable as coating materials. EUDRAGIT® is the trade name for copolymers derived from esters of acrylic and methacrylic acid, whose properties are determined by functional groups. The individual EUDRAGIT® grades differ in their proportion of neutral, alkaline or acid groups and thus in terms of physicochemical properties. The skillful use and combination of different EUDRAGIT® polymers offers ideal solutions for controlled drug release in various pharmaceutical and technical applications. EUDRAGIT® provides functional films for sustained-release tablet and pellet coatings. The polymers are described in international pharmacopeias such as Ph.Eur., USP/NF, DMF and JPE. EUDRAGIT® polymers can provide the following possibilities for controlled drug release: gastrointestinal tract targeting (gastroresistance, release in the colon), protective coatings (taste and odor masking, protection against moisture) and delayed drug release (sustained-release formulations). EUDRAGIT® polymers are available in a wide range of different concentrations and physical forms, including aqueous solutions, aqueous dispersion, organic solutions, and solid substances. The pharmaceutical properties of EUDRAGIT® polymers are determined by the chemical properties of their functional groups. A distinction is made between:
poly(meth)acrylates, soluble in digestive fluids (by salt formation) EUDRAGIT® L (Methacrylic acid copolymer), S (Methacrylic acid copolymer), FS and E (basic butylated methacrylate copolymer) polymers with acidic or alkaline groups enable pH-dependent release of the active ingredient. Applications: from simple taste masking via resistance solely to gastric fluid, to controlled drug release in all sections of the intestine.
poly(meth)acrylates, insoluble in digestive fluids: EUDRAGIT® RL and RS (ammonio methacrylate copolymers) polymers with alkaline and EUDRAGIT® NE polymers with neutral groups enable controlled time release of the active by pH-independent swelling.

Enteric EUDRAGIT® coatings provide protection against drug release in the stomach and enable controlled release in the intestine. The dominant criterion for release is the pH-dependent dissolution of the coating, which takes place in a certain section of the intestine (pH 5 to over 7) rather than in the stomach (pH 1-5). For these applications, anionic EUDRAGIT® grades containing carboxyl groups can be mixed with each other. This makes it possible to finely adjust the dissolution pH, and thus to define the drug release site in the intestine. EUDRAGIT® L and S grades are suitable for enteric coatings. EUDRAGIT® FS 30 D (aqueous dispersion of an anionic copolymer based on methyl acrylate, methyl methacrylate and methacrylic acid) is specifically used for controlled release in the colon.

In some embodiments, the food composition of the present invention is selected from complete food compositions, food supplements, and the like. The composition of the present invention may be used as a food ingredient and/or feed ingredient.

The food ingredient may be in the form of a solution or as a solid—depending on the use and/or the mode of application and/or the mode of administration.

The nutraceutical composition of the present invention may be solid, semi-solid or liquid. It may be in the form of a food product or food supplement, e.g. in the form of tablets, gels, powders, capsules, drinks, bars, etc. For example, the composition may be in the form of a powder packed in a sachet which can be dissolved in water, fruit juice, milk or another beverage.

In some embodiments, the drink is a functional drink or a therapeutic drink, a thirst-quencher or an ordinary drink. By way of example, the composition of the present invention can be used as an ingredient to soft drinks, a fruit juice or a beverage comprising whey protein, health teas, cocoa drinks, milk drinks and lactic acid bacteria drinks, yoghurt and drinking yoghurt, cheese, ice cream, water ices and desserts, confectionery, biscuits cakes and cake mixes, snack foods, balanced foods and drinks, fruit fillings, care glaze, chocolate bakery filling, cheese cake flavoured filling, fruit flavoured cake filling, cake and doughnut icing, instant bakery filling creams, fillings for cookies, ready-to-use bakery filling, reduced calorie filling, adult nutritional beverage, acidified soy/juice beverage, aseptic/retorted chocolate drink, bar mixes, beverage powders, calcium fortified soy/plain and chocolate milk, calcium fortified coffee beverage.

In some embodiments, the composition of the present invention is used with yoghurt production, such as fermented yoghurt drink, yoghurt, drinking yoghurt, cheese, fermented cream, milk-based desserts and others. Suitably, the composition can be further used as an ingredient in one or more of cheese applications, meat applications, or applications comprising protective cultures.

In some embodiments, the food composition of the present invention is suitable for preparing meal replacement product.

The food composition of the present invention typically comprises carriers or vehicles. Examples of nutritionally acceptable carriers include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like.

In some embodiments, the food composition of the present invention comprises an amount of dietary fibers. Dietary fibers pass through the small intestine undigested by enzymes and function as a natural bulking agent and laxative. Dietary fiber may be soluble or insoluble and in general a blend of the two types is preferred. Suitable sources of dietary fibers include soy, pea, oat, pectin, guar gum, gum Arabic, fructooligosaccharides, galacto-oligosaccharides, sialyl-lactose and oligosaccharides derived from animal milks. In some embodiments, the dietary fiber is selected among mannans. Mannans (such as glucomannans and galactomannans), such as guar gum, locust bean gum, konjac, and xanthan gum, are present in some plant cell walls. The glucomannans are generally comprised of (1-4)-β-linked glucose and mannose units, while the galactomannans are generally comprised of a (1-4)-β-mannan backbone substituted with single units of (1-6)-α-galactose. Many endospermic legumes, such as guar and locust bean, contain galactomannans in the endosperm during seed development. Glucomannans have also been found as a minor component of cereal grains.

In some embodiments, the food composition of the present invention contains minerals and micronutrients such as trace elements and vitamins in accordance with the recommendations of Government bodies such as the USDA or the EFSA. For example, the composition may contain per daily dose one or more of the following micronutrients in the ranges given: 300 to 500 mg calcium, 50 to 100 mg magnesium, 150 to 250 mg phosphorus, 5 to 20 mg iron, 1 to 7 mg zinc, 0.1 to 0.3 mg copper, 50 to 200 g iodine, 5 to 15 g selenium, 1000 to 3000 g beta carotene, 10 to 80 mg Vitamin C, 1 to 2 mg Vitamin B1, 0.5 to 1.5 mg Vitamin B6, 0.5 to 2 mg Vitamin B2, 5 to 18 mg niacin, 0.5 to 2.0 g Vitamin B12, 100 to 800 g folic acid, 30 to 70 g biotin, 1 to 5 g Vitamin D, and/or 3 to 10 g Vitamin E.

In some embodiments, the composition of the present invention contains emulsifiers. Examples of food grade emulsifiers typically include diacetyl tartaric acid esters of mono- and di-glycerides, lecithin and mono- and di-glycerides. Similarly, suitable salts and stabilizers may be included.

In some embodiments, the composition of the present invention contains protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellifying agents, gel forming agents, antioxidants and antimicrobials. The composition may also contain conventional pharmaceutical additives and adjuvants, excipients and diluents, including, but not limited to, water, gelatine of any origin, vegetable gums, ligninsulfonate, talc, sugars, starch, gum arabic, vegetable oils, polyalkylene glycols, flavouring agents, preservatives, stabilizers, emulsifying agents, buffers, lubricants, colorants, wetting agents, fillers, and the like. In all cases, such further components will be selected having regard to their suitability for the intended recipient.

In one embodiment, the composition, the pharmaceutical composition or the nutraceutical composition of the invention is formulated in an oral dosage form selected from enterically-coated tablets and enterically-coated capsules. In one embodiment, the enteric-coating enterically-coated of the formulation comprises or consists of a mixture of hydroxypropyl methyl cellulose and gellan gum. In one preferred embodiment, the oral dosage form is an enterically-coated capsule, wherein the enteric-coating is a mixture comprising hydroxypropyl methyl cellulose and gellan gum. In one embodiment, the enteric coating is the capsule itself. In one embodiment, the enteric coating comprises from 85 to 95% hydroxypropyl methyl cellulose and from 5 to 15% gellan gum (w/w) in weight relative to the enteric-coating or the capsule weight. In one embodiment, the enteric coating comprises about 95% hydroxypropyl methyl cellulose and about 5% gellan gum (w/w) in weight relative to the enteric-coating or the capsule weight. In one specific embodiment, the enteric-coating is a DRcaps™ capsule commercialized by Capsugel®.

In a last aspect, the invention relates to a blister, or preferably a container comprising the composition, the pharmaceutical composition, the food composition or at least one oral dosage form as previously described. Typically, the blister or preferably the container is moisture-tight. In one embodiment, the moisture-tight container comprises (a) a container body having a base and a sidewall extending therefrom, the container body defining an interior, the container body further having an opening leading to the interior and a lip surrounding the opening; (b) a lid being movable with respect to the container between a closed position in which the lid covers the opening so as to create a moisture tight seal with the container body and an open position in which the opening is exposed; (c) at least a first seal and a second seal, the first seal being formed by mating thermoplastic- to -thermoplastic sealing surfaces of the lid and the container body respectively, the first seal optionally including an undercut of the container body relative to a central axis of the container body or a lip seal member extending downward from the lid, the second seal being formed by mating elastomer-to-thermoplastic sealing surfaces, wherein the elastomer-to-thermoplastic sealing surfaces includes an elastomer formed in the lid or on the container body, optionally with multi-shot injection molding, wherein the thermoplastic is incompressible and the elastomer is compressible and optionally resilient; and optionally (d) an insert secured within the interior of the container body, the insert comprising a base material and a desiccant, wherein the base material provides structure to the insert and is optionally a polymer, the insert having an insert opening leading to an interior compartment configured for housing products. Advantageously, the container, when in the closed position, has a moisture vapor transmission rate, at ambient conditions of 30° C. and 75% relative humidity (RH), of less than 500 g/day, less than 400 g/day, less than 350 g/day, less than 325 g/day, less than 300 g/day, from 150 ag/day to 300 g/day, from 175 g/day to 285 g/day. In one specific embodiment, the moisture tight container is an Aptar CSP Technologies container such as for example Activ-vial™

Therapeutic and/or Nutraceutical Use of the Compositions

The present invention further relates to a composition according to the invention, preferably a pharmaceutical or nutraceutical composition according to the invention, for use in oxytocin-pathway related disorders, selected from autism and anxiety-related disorders.

In the context of the present invention, anxiety-related disorders may also refer to chronic-stress related disorders and are selected from anxiety-related depression and anxiety-related eating disorders that are selected from binge eating, sugar craving and anxiety-related weight gain, preferably selected from sugar craving and anxiety-related weight gain, even more preferably the anxiety-related eating disorder is sugar craving. Autism is an oxytocin-mediated disorder that is also encompassed among the disorders that may be treated according to the present invention. Autism is a developmental disorder characterized by difficulties with social interaction and communication, and by restricted and repetitive behavior. Thus, the composition according to the invention may be for use in the treatment of autism, or anxiety-related disorders selected from anxiety-related depression and eating disorders; preferably for use in the treatment of anxiety-related disorders selected from anxiety-related depression and anxiety-related eating disorders, in particular anxiety-related sugar craving, anxiety-related binge eating and the associated anxiety-related weight gain.

Depression refers to psychological states of aggressed mood characterized by sensations of sadness changes in mouth taste, body weight or sleep pattern, psychomotor agitation or retardation, accident ability, reduction in concentration or crystallinity, lack of activity and fatigue, emotion of unvaluable.

“Food addiction” or “food craving” refers to compulsive overeating that engages the subject in frequent episodes of uncontrolled eating (binge eating). In addition to binge eating, compulsive overwatering subjects may also engage in “grazing” behavior, during which they continuously eat throughout the day, thereby increasing the number of meals per day. These actions result in an excessive overall number of calories consumed, even if the quantities eaten at any one time may be substantially unchanged compared to the quantities consumed by the average population or the subject prior to his food addiction.

Sugar craving is a specific type of food addiction wherein the compulsive eating is directed to high-sucrose food compositions. By treating binge eating or sugar craving is also meant the treatment or the attenuation of the associated symptoms selected from weight-gain, sleeping troubles such as oversleeping, restlessness, irritability, digestive disorders, and headaches, preferably selected from weight-gain.

Thus, in one embodiment, the composition is for use in the treatment of anxiety-related disorders selected from anxiety-related depression and anxiety-related eating disorders, in particular anxiety-related sugar craving and/or anxiety-related binge eating and the associated anxiety-related weight gain.

All embodiments listed above regarding the pharmaceutical compositions of the invention apply mutatis mutandis to the composition for use according to the invention.

The present invention further relates to the use of a composition as described above in the manufacture of a medicament for the treatment of oxytocin-pathway related disorders, selected from autism and anxiety-related disorders as detailed above.

The present invention further relates to method for the treatment of autism, or anxiety-related disorders selected from anxiety-related depression and eating disorders, as described above, comprising administering to a subject in need thereof a therapeutically and/or nutraceutically effective amount of a composition according to the invention.

The compositions to be used according to the invention are preferably therapeutical and/or nutraceutical compositions according to the invention.

The posology may vary within a wide range depending on the therapeutic and/or nutraceutical indication and the route of administration, and also the general health, age, sex, and body weight of the individual. The skilled artisan will be able to determine appropriate dosages depending on these and other factors.

For example, when the composition according to the invention is administered as a pharmaceutical composition, it may typically comprise a total amount of strains ranging from 10⁵ to 10¹⁵ CFU per gram of therapeutical composition.

When the composition according to the invention is administered as a nutraceutical composition, for instance as a food product, it may typically comprise a total amount of strains ranging from 10³ to 10¹² CFU per gram of nutraceutical composition, preferably per gram of food product.

In some embodiments, the administration of the composition according to the invention, preferably of the pharmaceutical and/or nutraceutical composition according to the invention, is repeated, for example, 2 to 3 times a day, for one day or more and generally for a sustained period of at least 4 days, or even 4 to 15 weeks, with, where appropriate, one or more periods of interruption.

In some embodiments, the composition according to the invention, preferably of the pharmaceutical and/or nutraceutical composition according to the invention, is administered simultaneously or sequentially to a meal of the subject. In some embodiments, the composition according to the invention is administered prior to the meal of the subject.

In some embodiments, the pharmaceutical compositions according to the invention are administered in combination with at least another active agent, preferably another active agent in the treatment of mood disorders, for instance an anti-depressive agent.

The combination of the pharmaceutical composition of the invention with at least one other active agent may be simultaneous, sequential or separate.

In one embodiment, the at least one other active agent may be at least one selective serotonin reuptake inhibitor and/or at least one serotonin-norepinephrine reuptake inhibitor. In one embodiment, the at least one selective serotonin reuptake inhibitor is selected from the group consisting of citalopram, escitalopram, fluoxetine, fluvoxamine, paroxetine, and sertraline. In one embodiment, the at least one serotonin-norepinephrine reuptake inhibitor is selected from the group consisting of atomoxetine, desvenlafaxine, duloxetine, levomilnacipran, milnacipran and venlafaxine.

### Peptide Compositions

The invention finally relates to a composition comprising a mixture of bioactive proteins or peptides comprising at least one bioactive protein or peptide that is able to cross-react with anti-oxytocin antibodies and/or presents an oxytocin-like effect. The invention particularly refers to the use of such in the treatment of autism or anxiety related disorders selected from depression and eating disorders as described above.

To the inventors surprise the peptides from a L. salivarius strain and a L. gasseri strain are not only conformational mimetics of oxytocin but also comprise segments that directly activate the oxytocin-receptor.

The invention relates to a composition comprising a mixture of bioactive proteins or peptides comprising at least one, preferably at least four, even more preferably at least five bioactive proteins or peptides. It can be understood that the mixture of bioactive peptides may be a mixture of synthetic peptides or a mixture deriving from attenuated, lysed and optionally fractionated L. salivarius and L. gasseri probiotic composition. Thus, the mixture of bioactive peptides may be a mixture of at least one, preferably, at least four, even more preferably at least five proteins of from SEQ ID NO:1, SEQ ID NO:2, SEQ ID NO:3, SEQ ID NO:4, SEQ ID NO:5, SEQ ID NO:6, SEQ ID NO:7, SEQ ID NO:8 or bioactive peptides (fragments) thereof as described above.

According to a preferred embodiment, the at least one bioactive peptide is selected from a at least one peptide presenting from at least 80%, preferably from at least 85%, from at least 90%, even more preferably from at least 95%, or 100% identity with any one of the following peptides:

SEQ ID 28: H-DYVKNMITG-OH,
SEQ ID NO 29: H-KFVNDNELG-OH,
SEQ ID NO 30: H-KTVVWNGPMG-OH,
SEQ ID NO 33: H-QYIQEGR-OH,
SEQ ID NO 35: H-DYIKNMITG-OH,
SEQ ID NO 36: H-VDIQEFMIMPVG-OH,
SEQ ID NO 37: H-SYFYNKEDG-OH,

In particular, the composition comprises a mixture of bioactive peptides comprising at least one, preferably at least four, even more preferably at least five bioactive peptides, wherein the at least one bioactive peptide is selected from the group consisting of peptides presenting from at least 80%, from at least 85%, preferably from at least 90%, even more preferably from at least 95%, or 100% identity with any one the peptides of SEQ ID 28, SEQ ID NO 29, SEQ ID NO 30, SEQ ID NO 33, SEQ ID NO 35, SEQ ID NO 36, and SEQ ID NO 37, for use in the treatment of autism or anxiety related disorders selected from depression and eating disorders. In one embodiment, the composition for use in the treatment of autism or anxiety related disorders selected from depression and eating disorders comprises a mixture of bioactive peptides comprising at least one, preferably at least four, even more preferably at least five bioactive peptides, wherein the at least one bioactive peptide is selected from peptides presenting at least 95%, or 100% identity with any one the peptides of SEQ ID 28, SEQ ID NO 29, SEQ ID NO 30, SEQ ID NO 33, SEQ ID NO 35, SEQ ID NO 36, and SEQ ID NO 37, typically the at least one bioactive peptide being selected from peptides of SEQ ID 28, SEQ ID NO 29, SEQ ID NO 30, SEQ ID NO 33, SEQ ID NO 35, SEQ ID NO 36, and SEQ ID NO 37.

According to one embodiment, the composition comprises a mixture of bioactive peptides consists of at least one, preferably at least four, even more preferably at least five bioactive peptides as described above.

The composition comprising at least one bioactive peptide according to the invention may comprise all the same (non-probiotic) components as descried above for the compositions comprising the strain combinations, apart from the strains themselves.

The composition comprising at least one bioactive peptide according to the invention may be used as disclosed above for nutraceutical and/or pharmaceutical applications.

In particular, the invention relates to a bioactive peptide or a pharmaceutical composition comprising at least one bioactive peptide according to the invention for use in the treatment of at least one neuropsychiatric disorder, such as anxiety, depression and/or autism. In one specific embodiment, the bioactive peptide or the pharmaceutical composition according to the invention is for use in the treatment of depression.

The invention also relates to the use of a bioactive peptide or a nutraceutical composition comprising at least one bioactive peptide according to the invention in the management of mood disorders. The bioactive peptide or a nutraceutical composition comprising at least one bioactive peptide according to the invention is preferably used for promoting, maintaining and/or improving comfort or for alleviating and/or preventing a discomfort in a subject suffering from at least one mood disorder or any symptom thereof.

In some embodiments, the invention relates to the use of a bioactive peptide or a nutraceutical composition comprising at least one bioactive peptide according to the invention for alleviating stress or anxiety, or any symptom thereof. In some embodiments, the bioactive peptide or the nutraceutical composition comprising at least one bioactive peptide according to the invention is used for improving the mood and/or for relieving the every-day stress of a healthy subject.

All embodiments and features disclosed above for the compositions comprising strain combinations according to the invention and for their therapeutical and/or nutraceutical use apply similarly to the bioactive peptide or composition comprising at least one bioactive peptide according to the invention.

### BRIEF DESCRIPTION OF THE DRAWINGS

FIG. 1 is a diagram representing the experimental design of Example 1.

FIG. 2 is a histogram showing the effect of Mix 1 treatment in Open-field test in mice before (DO) and 10 days after (D11) gavage with a mix of L. salivarius and L. gasseri (LS+LG) or with 0.9% NaCl solution as control (Control). Total distance traveled (a) and central to peripheral time ratios (b), in the open-field test. *p<0.05, **p<0.01, paired t-tests.

FIG. 3 is a histogram showing the effect of Mix 1 treatment in O-maze test during chronic mild stress (CMS) in mice receiving a mix of L. salivarius and L. gasseri (LS+LG) or 0.9% NaCl solution as control (Control). Distance moved in open (a) and closed (b) arms as well as time spent in open (c) and closed (d) arms of the O-maze.

FIG. 4 is a histogram showing the effect of Mix 1 treatment on tail-suspension test (TST) during chronic mild stress (CMS) in mice receiving a mix of L. salivarius and L. gasseri (LS+LG) or 0.9% NaCl solution as control (Control). Immobility time (a) and escape energy (b) in the TST. RU, relative units, b, Two-way RM ANOVA, p=0.05, *p<0.05 Bon-ferroni post-tests.

FIG. 5 is a histogram showing the effect of Mix 1 treatment forced-swim test (FST) during the CMS in mice receiving a mix of L. salivarius and L. gasseri (LS+LG) or 0.9% NaCl solution as control (Control), expressed as dynamics. a. Two-way RM ANOVA, effect of treatment p=0.2, *p<0.05, Stu-dent's t-test.

FIG. 6 is a graph showing the effect of Mix 1 on forced-swim test (FST) during the CMS in mice receiving a mix of L. salivarius and L. gasseri (LS+LG) or 0.9% NaCl solution as control (Control), expressed as total immobility time in the FST. Two-way RM ANOVA, effect of treatment p=0.2, *p<0.05, Student's t-test.

FIG. 7 is a graph showing the effect of Mix 1 on Body weight dynamics (a) and characteristics of ingestive behavior during the CMS (b-h) in mice receiving a mix of L. salivarius and L. gasseri (LS+LG) or 0.9% NaCl solution as control (Control). Water intake (b), food intake (c), meal size (d), meal number (e), total daily intake of 2% sucrose (f), volume of each 2% sucrose intake (g) and number of 2% sucrose intakes per day (h). **p<0.01, ***p<0.001, Student's t-tests

FIG. 8 is a graph showing the presence of OT-like immunoreactivity in bacterial protein homogenates of L. salivarius, L. gasseri and the Enterobacteriaceae taxon. ANOVA p<0.01, *p<0.05, **p<0.01, Tukey's-post-tests

### EXAMPLES

The present invention is further illustrated by the following examples.

### Example 1: Screening of Bacterial Strains—Proteomic Analysis

The present study aims at screening Lactobacilli and Bidobacteria for their capacity of their components to present oxytocin-like effects.

### Materials and Methods

In this study, 14 bacteria-extracted proteomes were analyzed: (i) from 5 Bifidobacterium species grown in anaerobe conditions, (ii) from 5 Lactobacillus species grown in anaerobe culture conditions and (iii) from 4 aerotolerant Lactobacillus species grown in aerobe conditions. Each proteome analysis was performed in triplicate and was further separated in 3 fractions i.e. soluble (S), membrane (M), and residual (R) resulting in 9 samples/proteome). Each fraction was initially screened by western blot (WB) for the presence OT-like reactivity to validate it for further 2D-SDS-PAGE analysis. The WB revealed OT-like immunoreactive bands in most but not all samples (data not shown). Finally, all fractions have been analyzed using 2D gel technique and OT immunodetection. 2D gel electrophoresis allowed to improve visibility of the less abundant proteins in a complex sample. Thereby, all samples were analyzed at least once with this method to scan optimally each proteome in the search for OT mimicry. By this way, some samples for which no OT-immunoreactivity had been identified by 1D-western blot, showed positive results by 2D-immunoblotting. Samples exhibited positive immunoreactivity with OT IgG were analyzed in duplicate. The specificity for OT-like staining was controlled by pre-adsorption of OT-IgG with OT peptide.

All protein spots in silver nitrate staining gels corresponding to OT-like spots in the membrane were excised and analyzed by mass spectrometry

### Results

The proteomic analysis revealed 44 target proteins: 27 in Lactobacillus and 17 in Bifidobacterium, variably distributed in all samples. Among them, 7 were common to both genus: phosphoglycerate kinase (PGK), elongation factor thermo unstable (EFTU), enolase and 3 chaperone proteins. Conversely, some target proteins were specific to the genus or to a strain.

Target proteins were first selected based on their alignment score with the OT sequence. Then, they were sorted according to their recurrence (i) between all samples for common proteins, (ii) between strains for a genus-specific protein and (iii) between replicates for a strain-specific protein. The 60 kDa chaperonin protein was eliminated from 7 common target proteins despite its high recurrency between samples because the molecular weight of the spot did not always match, which may due to contamination of the target spot.

The PGK (phosphoglycerate kinase), EFTU (elongation factor tu) and ENO (enolase) have been found to represent common proteins group. In fact, PGK was identified in 5 strains (2 Lactobacillus and 3 Bifidobacterium), EFTU in 7 strains (5 Lactobacillus and 2 Bifidobacterium) and ENO in 5 strains (2 Lactobacillus and 3 Bifidobacterium). The glucose-6-phosphate isomerase (G6PI) was identified in LS (L. salivarius), LG (L. gasseri) and LR (L. reuteri) strains i.e. was Lactobacillus genus specific. Bifidobacterium group was represented by a phosphoketolase protein identified in BR (B. ruminantium), BD (B. dentis) and BPG (B pseudolongum subsp. globosum) strains. Proteomes obtained from LP (L. plantarum) and LC (L. camelliae were different from the other strains. Proteomic analysis revealed respectively 2 and 4 representative targets of these 2 distinctive strains. Although the common and Lactobacillus-specific targets are highly conserved in LP and LC, they were not or poorly identified in these strains. Similarly, the GMP synthase (GUAA) and the aminotransferase (GLMS) which represented LP, as well as the aldolase, the reductase, the isomerase and the mutase found in LC, were not identified in other Lactobacilli. These 6 proteins were identified as LP or LC-specific targets (Table 1).

The inventors found out that the following Lactobacillus and Bifidobacterium strains express the following proteins that surprisingly immune cross-react with anti-oxytocin antibodies and thus present an oxytocin-like effect:
Lactobacillus salivarius:
   Elongation factor Tu having an amino acid sequence of SEQ ID NO:1,
   Glucose-6-phosphate isomerase having an amino acid sequence of SEQ ID NO:2, and
   Phosphoglycerate kinase having an amino acid sequence of SEQ ID NO:3.
Lactobacillus gasseri:
   Elongation factor Tu having an amino acid sequence of SEQ ID NO:4,
   Glucose-6-phosphate isomerase having an amino acid sequence of SEQ ID NO:5,
   Phosphoglycerate kinase having an amino acid sequence of SEQ ID NO:6,
   Enolase 1 having an amino acid sequence of SEQ ID NO:7, and
   Enolase 2 having an amino acid sequence of SEQ ID NO:8,
Lactobacillus camelliae:
   Elongation factor Tu having an amino acid sequence of SEQ ID NO:9,
   Fructose tagatose bisphosphate aldolase having an amino acid sequence of SEQ ID NO:10,
   2,5-didehydrogluconate reductase having an amino acid sequence of SEQ ID NO:11,
   Triosephosphate isomerase having an amino acid sequence of SEQ ID NO:12, and
   2,3-bisphosphoglycerate-dependent phosphoglycerate mutase having an amino acid sequence of SEQ ID NO:13,
Lactobacillus plantarum:
   Elongation factor Tu having an amino acid sequence of SEQ ID NO:14,
   GMP synthase having an amino acid sequence of SEQ ID NO:15, and
   Glutamine-fructose-6-phosphate aminotransferase having an amino acid sequence of SEQ ID NO:16.
Lactobacillus reuteri:
   Elongation factor Tu having an amino acid sequence of SEQ ID NO:17, and
   Glucose-6-phosphate isomerase having an amino acid sequence of SEQ ID NO:18.
Bifidobacterium dentium:
   Phosphoglycerate kinase having an amino acid sequence of SEQ ID NO:19, and
   Phosphoketolase having an amino acid sequence of SEQ ID NO:20.
Bifidobacterium pseudolongum subsp. pseudolongum:
   Elongation factor Tu having an amino acid sequence of SEQ ID NO:21, and
   Phosphoglycerate kinase having an amino acid sequence of SEQ ID NO:22.
Bifidobacterium pseudolongum subsp. globosum:
   Phosphoglycerate kinase having an amino acid sequence of SEQ ID NO:23, and
   Prolable phosphoketolase having an amino acid sequence of SEQ ID NO:24.
Bifidobacterium ruminantium:
   Phosphoketolase having an amino acid sequence of SEQ ID NO:25.
Bifidobacterium adolescentis:
   Elongation factor Tu having an amino acid sequence of SEQ ID NO:26.

Based on the results on OT-like protein identification, 17 proteins were selected for the synthesis of target peptide fragments. These peptides corresponded to the part of the target sequences which aligned with the OT sequence. Altogether, 17 potential OT-like peptides derived from the proteomic analysis have been selected and synthesized (Table 1).

| | | | | SEQ | Bacterial |
|---|---|---|---|---|---|
| #Id. | Protein name | Peptide Sequence | Position | ID NO: | origin |
| 1 | Elongation factor Tu | H-DYVKNMITG-OH | 87-95 | 28 | LS, LR, |
| | | | | | LP, LC |
| 2 | Glucose-6-phosphate | H-KFVNDNELG-OH | 13-21 | 29 | LS |
| | isomerase | | | | |
| 3 | Phosphoglycerate kinase | H-KTVVWNGPMG-OH | 317-326 | 30 | LG, LS, BD, |
| | | | | | BPP, BPG |
| 4 | GMP synthase | H-ATSKNCPIA-OH | 154-162 | 31 | LP |
| | [glutamine-hydrolyzing] | | | | |
| 5 | Glutamine-fructose-6- | H-LTITNVPNS-OH | 371-379 | 32 | LP |
| | phosphate | | | | |
| | aminotransferase | | | | |
| | [isomerizing] | | | | |
| 6 | Glucose-6-phosphate | H-QYIQEGR-OH | 313-319 | 33 | LG |
| | isomerase | | | | |
| 7 | Glucose-6-phosphate | H-KQFVHENELG-OH | 12-21 | 34 | LR |
| | isomerase | | | | |
| 8 | Elongation factor Tu | H-DYIKNMITG-OH | 88-96 | 35 | LG |
| 9 | Enolase 2 | H-VDIQEFMIMPVG-OH | 160-171 | 36 | LG |
| 10 | Enolase | H-SYFYNKEDG-OH | 246-254 | 37 | LG |
| 11 | Fructose tagatose | H-MFQAARKG-OH | 1-8 | 38 | LC |
| | bisphosphate aldolase | | | | |
| 12 | 2,5-didehydrogluconate | H-AYSPLGTGK-OH | 202-210 | 39 | LC |
| | reductase | | | | |
| 13 | Triosephosphate isomerase | H-CYFEDAGA-OH | 61-68 | 40 | LC |
| 14 | 2,3-bisphosphoglycerate- | H-KYIENISDE-OH | 191-199 | 41 | LC |
| | dependent phosphoglycerate | | | | |
| | mutase | | | | |
| 15 | Probable phosphoketolase | H-IYLRSNPLMK-OH | 40-49 | 42 | BR, BD, BPG |
| 16 | Elongation factor Tu | H-GFDRDCPVV-OH | 164-172 | 43 | BPP (locus B) |
| 17 | Elongation factor Tu | H-DFVKNMITG-OH | 89-97 | 44 | BPP (locus L) |

Table 1 showing the OT-like peptide fragments derived from the proteomic analysis

### Example 2: In Vitro Activation of Oxytocin Receptor (OT-R) by Oxytocin-Like Bacterial Peptide Fragments

The present example studies the direct effect of peptides of SEQ ID NO:28 to SEQ ID NO: 44 on the Oxytocin receptor.

### Materials and Methods

The activation of OT-R by bacterial peptides was analyzed by the measurement of calcium release from HEK cells transfected with human OT-R

### Results

As a negative control, no activation of non-transfected HEK cells with OT or bacterial peptides was observed. In contrast, using OTR expressing HEK cells, OT and out of 17 OT-like bacterial peptides have specifically activated OTR. Among these peptides there were: (i) 4 peptides (#1, 3, 7 and 8) from common proteins (PGK and EFTU), (ii) 3 peptides from Lactobacillus-specific proteins (#2, #6 and #7 as G6PI), 2 peptides from LP-specific proteins (#4 as GUAA and #5 as GLMS) and 1 peptide from LC-specific proteins (#14 as GPMA).

These results revealed that peptides derived from bacterial proteins specifically activate OTR even if their affinity is about 200-500 times lower than the OT peptide itself. Indeed, all these peptides have various micromolar EC₅₀ compared to the nanomolar EC₅₀ of OT. This characteristic and the maximal amplitude of Ca²⁺ release allow to highlight most efficient OT-like peptides ex. #1 (EFTU) and #3 (PGK). The only one common strain for both of these peptides, LS, was selected for in vivo studies. On the other hand, all synthetized peptides from EFTU (L locus) also specifically activated OTR. Because both target proteins EFTU and PGK were identified in LG, this strain was selected to be added to the mix with LS. Even if these two targets have been identified in BP as in LG, the EFTU sequence modifications in Bifidobacterium (L locus) reduced activity on OTR.

TABLE 2
Table showing the EC₅₀ and Emax of each peptide
as mean ± standard error (ND for not determined).

| SEQ | | | |
|---|---|---|---|
| ID NO: | #Id | EC₅₀ | Emax |
| 28 | 1 | 2.77 × 10⁻⁶ ± 0.79 | 273.8 ± 35.03 |
| 29 | 2 | 3.21 × 10⁻⁶ ± 1.85 | 164.8 ± 107.9 |
| 30 | 3 | 8.62 × 10⁻⁶ ± 0.50 | 302 ± 35.41 |
| 31 | 4 | 3.09 × 10⁻⁶ ± 0.48 | 304.6 ± 29.70 |
| 32 | 5 | 2.70 × 10⁻⁶ ± 1.03 | 272 ± 99.09 |
| 33 | 6 | 3.79 × 10⁻⁶ ± 0.73 | 99.42 ± 6.77 |
| 34 | 7 | 2.57 × 10⁻⁶ ± 0.13 | 262.8 ± 35.64 |
| 35 | 8 | 2.80 × 10⁻⁶ ± 1.23 | 140 ± 71.48 |
| 36 | 9 | nd | 119.4 ± 47.19 |
| 37 | 10 | nd | 41.63 ± 32.19 |
| 38 | 11 | nd | 4.65 ± 2.59 |
| 39 | 12 | nd | 5.25 ± 0.97 |
| 40 | 13 | nd | 17.92 ± 0.66 |
| 41 | 14 | 8.93 × 10⁻⁶ ± 8.16 | 221.5 ± 10.40 |
| 42 | 15 | nd | 5.36 ± 0.31 |
| 43 | 16 | nd | 2.13 ± 0.83 |
| 44 | 17 | 7.52 × 10⁻⁶ ± 0.10 | 135.3 ± 8.92 |
| 27 | OT | 1.09 × 10⁻⁹ ± 0.19 | 253.77 ± 5.25 |

### Example 3: In Vivo Effects of Mix 1 on Anxiety, Depression-Like Behavior, Ingrestive Behavior and Body Weight

The present study aims at determining the effects of a Lactobacilli mixture, Mix 1, on the anxiety, depression-like behavior, ingestive behavior and body weight in mice during chronic mild stress.

### Materials and Methods

The effects of a Lactobacilli mixture (Mix 1) comprising Lactobacillus Salivarius and Lactobacillus Gasseri at 10¹⁰ CFU each was assessed in vivo as follows.

### Bacterial Cultures

Strains of Lactobacillus, the L. salivarius LS7892 (DSM 16530) and L. gasseri LG6410 (LMG P-29638) were produced by CSL/Sacco (Zelo Buon Persico, Italy) and provided by TargEDys SA (Longjumeau, France). Both L. salivarius and L. gasseri bacteria were cultivated overnight in 20 mL of Man Ragosa Sharp broth (MRS, Difco) at 37° C. with shaking (140 rpm). The resulting culture was diluted at optical density 600 nm (OD600) of 0.01 (˜2×10⁶ CFU/mL) in 200 mL of MRS and incubated for 24 h at 37° C. with shaking (140 rpm). Each culture was performed in triplicate. Additionally, for the detection of OT-like reactivity in taxonomically distant from Lactobacillus commensal bacteria, Escherichia coli K12 and Hafnia alvei have been cultured as previously described (Legrand R, Lucas N, Dominique M, Azhar S, Deroissart C, Le Solliec M-A, et al. Commensal Hafnia alvei strain reduces food intake and fat mass in obese mice-a new potential probiotic for appetite and body weight management. Int J Obes. 2020; 44(5):1041-51); they are presented here together as Enterobacteriaceae. For the animal experiments we used lyophilized Lactobacillus bacteria. In brief, 200 mL of 24 h-culture were centrifuged at 3000 g during 20 min at 4° C., pellets were washed 3 times with PBS 0,1M, pH 7.4 and freezed overnight at −80° C., then lyophilized for 24 h (Alpha 2-4 LSCbasic, Christ, Osterode am Harz, Germany). Lyophilized bacterial powder was aliquoted by a 30 mg sample. Every day prior to gavage, bacterial aliquote was resuspended in 4 mL of NaCl 0.9%.

### Animals

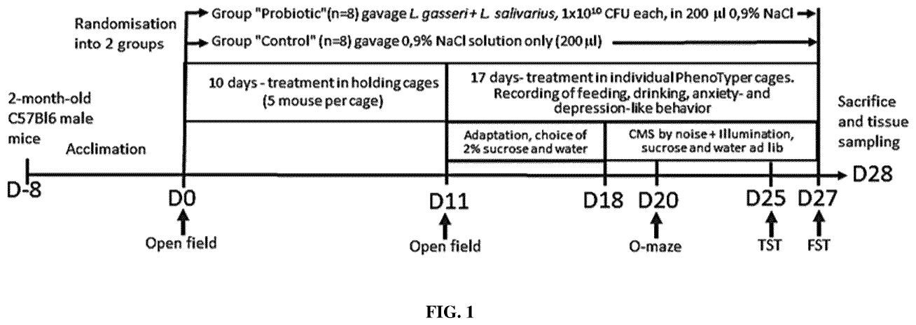

2-months old male C57Bl/6 mice were purchased from Janvier Labs (Le Genest-Saint-Isle, France) and were housed in individually ventilated standard holding cages for mice (Techniplast, Buguggiate, Italy), 5 animals per cage, with ad libitum access to a pelleted standard diet (SERLAB, Montataire, France) and drinking water. Mice were kept at 22±1° C. under a 12-h light/12-h dark cycle (light on between 07:00 h and 19:00 h). After 1-week acclimation to the animal facility, mice were randomly assigned to one of 2 study groups (n=8 each, after elimination of 1 mouse with a lowest body weight per holding cage). The treatment group received the probiotic mix of L. salivarius and L. gasseri (LS+LG), 1×10¹⁰ CFU each in 200 μl of 0.9% NaCl solution via intragastric gavage using a special drenching cannula for mice (Socorex, Ecublens, Switzerland). The control group received by intragastric gavage 200 μl of 0.9% NaCl solution. Considering the potential use of probiotics in the general population exposed to chronic stress, in contrast to medication used for treatment of anxiety and depression diseases, in the present CMS (chronic mild stress) protocol, we did not aim to create strictly controlled increase in anxiety and depression, which explains the absence of the CMS-free groups of mice. The procedures of intragastric gavage and body weight measures were done daily between 09:00 h and 10:00 h throughout the duration of the experiment. After 10 days of treatment in holding cages, mice were placed individually into the Modular Phenotypic cages, described below. After 1 week of adaptation to Modular Phenotypic cages, mice were submitted to the CMS model consisting of intermittent sound and light: white noise of 85 DB and illumination during 3 h, 08:00-11:00 PM. Ingestive behavior, including food intake, and 2% sucrose intakes were automatically monitored using a BioDAQ system (see below). Water intake was measured manually. During the CMS, food, water and 2% sucrose solution were available ad libitum, while during the adaptation period access to water and 2% sucrose were alternated daily. Mice underwent several behavioral tests for the analysis of locomotor, anxiety and depressive-like behavior on several days as shown in the FIG. 1. The behavioral tests are described below in details. Animal experimentation was performed by authorized investigators according to the European Community Council Directive of Nov. 24th 1986 (86:609: EEC) and the project was approved by the regional ethical committee CENOMEXA, certified by the Ministry of Research n° 54, Agreement n°: #8690.

### Behavioral Tests

### 1) Open-Field

Mice were isolated for 15 min in individual cages before being placed in the experimental device (40 cm×40 cm×30 cm) for 1 h with a light intensity of 100 lux and recorded using a computerized actimeter (Omnitech Electronics Ins., Columbus, OH, USA). The total distance moved was analyzed to evaluate exploratory activity. Anxiety behavior was estimated by distance moved and time spent in central zone (30 cm×30 cm) of the open-filed.

### 2) Elevated 0-Maze

The elevated-O-maze was performed as previously described [53]. Briefly, mice were placed into one of the two closed segments (surrounded by two infrared black walls 27 cm high) which constitute the circular O-maze platform (2 open arms/2 closed arms, diameter 45 cm, width 6 cm, elevated 60 cm). The distance moved and the time spent in each arm were measured for 5 min using a video camera placed above the O-maze and the EthoVision video tracking software XT v15.0 (Noldus I T, Wageningen, The Netherlands).

### 3) Tail Suspension Test (TST)

The device BioTST (BIOSEB, Vitrolles, France) consists of a black and white chlorure polyvinyle suspension unit of 50 cm×15 cm×30 cm. After placing in individual cages for 15 min, mice were suspended for 6 min by the tail using a tape placed on the suspension hook at the top of the device. The hook was connected to TST-SOFT software (BIOSEB, Vitrolles, France) which recorded mice activity time as well as energy and power deployed to escape.

### 4) Forced Swim Test (FST)

The forced swimming test (FST, [54]) was performed in a larger Plexiglas cylinder (height 25 cm, diameter 17 cm) containing 17 cm of water at 25±1° C. After placing in individual cages for 15 min, the immobility time of mice were measured for 6 min using an automated device for FST with video camera placed above the device and the Bio-FST software (FST X'PERT, BIOSEB, Vitrolles, France).

### 5) Modular Phenotypic Cages

The modular phenotypic cage is a device designed at the SCAC platform (University of Rouen Normandy, France), by coupling to a modified PhenoTyper (Noldus Information Technologies) a BioDAQ food and drink intake monitor with automated gate controller (BIODAQ E3, Research Diets, Inc., New Brunswick, NJ, USA). The PhenoTyper is an instrumented observation Plexiglas cage (30 cm×30 cm×35 cm) equipped with a top unit containing an infrared sensitive camera with three arrays of infrared LED lights to adjust light conditions and follow mice as well as an audio stimulus system controlled with ETHOVISION XT software. A 10 cm×10 cm×5 to 6 cm grey shelter was added into the PhenoTyper. Food and drink intake data were analyzed using the BioDAQ data viewer 2.3.09 software. This device was used to monitor locomotor activity, anxiety-like behavior, food, water, and sucrose intakes.

### 6) Monoamine Concentrations in the Brain

Next day after the FST, mice were killed and the brains were extracted and frozen in dry ice for the further analysis of monoamine concentration by ultrahigh-performance liquid chromatography (UHPLC) in tissue homogenates. In details, microdissections of different brain regions were performed based on mouse brain atlas [55]. The bilateral samples of the corpus striatum, nucleus accumbens, prefrontal cortex, hippocampus, amygdala and hypothalamus were weighed and sonicated in 0.12% of perchloric acid. After centrifugation for 60 min at 18 000 g, the supernatants were collected and aliquots were used for analysis of 5-HT, DA and L-DOPA by UHPLC. The UHPLC analysis was performed using a C18 MG100 reversed-phase column (100 mm×1.5 mm, 3 m, Shiseido Co., Ltd., Tokyo, Japan) under isocratic conditions with electrochemical detection. The mobile phase consisted of 75 mM NaH2PO4, 4.76 mM citric acid, 3 mM sodium dodecyl sulfate, 50 μM EDTA, 10% (v/v) methanol and 15% (v/v) acetonitrile (pH 5.62). Electrochemical detection was achieved by setting a glassy carbon working electrode at +600 mV in the Amperometric Analytical Cell Model 6041RS and at +650 mV in the 6020RS Coulometric Cell (ThermoFisher Scientific). Concurrently run standards, containing each of the neurotransmitters (Sigma), were used to calculate the concentration of each neurochemical. Concentrations of monoamines are shown as pg/μg of tissue, using an external calibration curve.

### 7) Statistical Analysis

Data were analyzed and the graphs were plotted using the GraphPad Prism 5.02 (GraphPad Software Inc., San Diego, CA). Data are shown as means±standard error of means (SEM), and for all tests p<0.05 was considered statistically significant.

### Results

### 1) Open-Field

The open-field test was performed at day 0 (DO) i.e., the day before starting the gavage procedure and it was repeated after 10 days of gavage at day 11 (D11), just before placing the mice into the individual Modular Phenotypic cages and without applying any CMS conditions (FIG. 1).

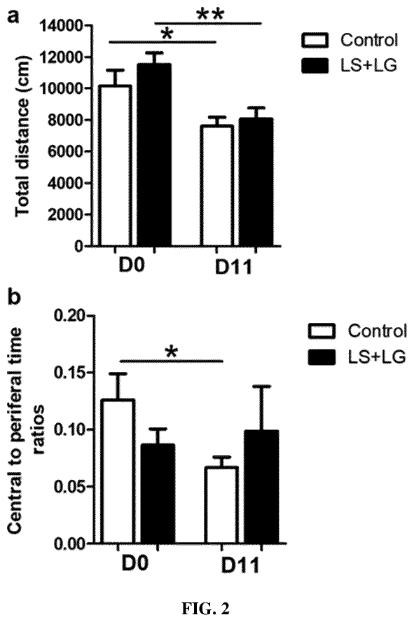

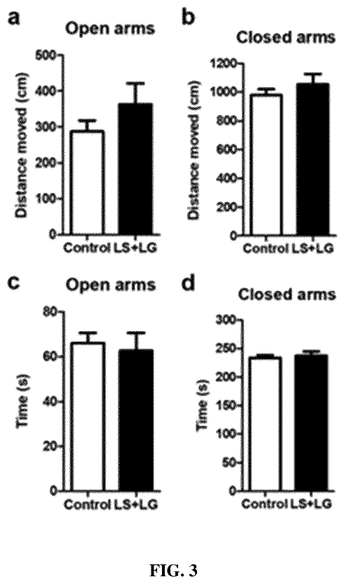

A decrease of locomotor activity at D11 vs. DO was observed in mice from both groups, but it was not significantly different between the groups either before or after gavage (FIG. 2a). Because elevated anxiety-like behavior in the open-field test is witnessed by reduction of the time spent in the center of the arena, we compared the central to peripheral time ratios. It was found that such ratios were not significantly different between the groups either at DO or D11; a decrease of such ratios was however, observed in the control but not in probiotic-treated group in D11 vs. DO (FIG. 2b).

Thus, the open-field results show a trend of an increase of anxiety in the control but not in the probiotic-treated mice after 10 days of gavage.

### 2) O-Maze

The O-maze test for evaluation of anxiety due to natural fears of rodents for height and open spaces was performed at day 20, i.e. 2 days after beginning of the CMS protocol (FIG. 1). The group of mice receiving the probiotic mix showed a tendency of increased moved distance as compared to the controls, but this difference did not reach significance in the present test.

### 3) Tail-Suspension Test (TST)

The TST test for evaluation of depressive-like behavior was performed at day 25, i.e. during the 6th day of the CMS protocol (FIG. 1). The group of mice receiving the probiotic mix showed a tendency of decreased immobility in the TST as compared to the controls, with a reduction of total immobility time by 25%, FIG. 4a). Furthermore, the probiotic group displayed a significant increase of the escape energy (FIG. 4b).

### 4) Forced-Swim Test (FST)

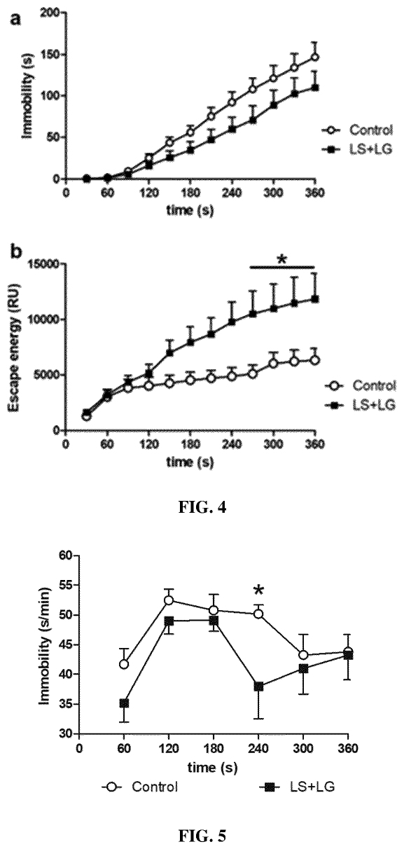

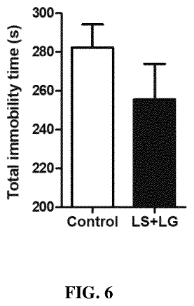

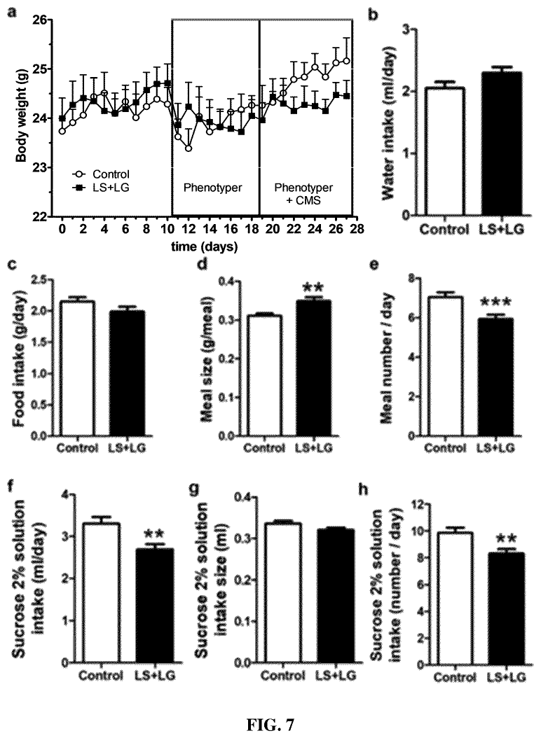

The FST test for evaluation of depressive-like behavior was performed during the CMS protocol 2 days later after the TST test (FIG. 1). Time of immobility, as an estimate of depressive-like behavior, showed an overall tendency of a reduction in the probiotic-treated group, with a significant decrease during the 4th min (FIG. 5a). The total immobility time showed a tendency of reduction by about 10% in the probiotic group (FIG. 6).

### 5) Modular Phenotypic Cages

During the CMS (D19-D27) a trend of more pronounced increase of body weight in the control group compared to the probiotic group was visible.

Daily water and food intakes during the CMS were not significantly different between the groups (FIG. 7b,c, Student's t-tests p=0.09, and p=0.13, respectively). However, the ratios of food to water intake were lower in the probiotic vs. the control group (0.87±0.05 vs. 1.07+0.06 g/ml, respectively, Student's t-test p<0.05).

The feeding pattern was significantly affected by the treatment, showing an increase of meal size but a decrease of meal number in the probiotic group (FIG. 7d,e).

Interestingly, during the CMS, the total daily intake of 2% sucrose solution was lower in the probiotic-treated group (FIG. 7f). This decrease was mainly due to a reduction of number of intakes, because the size of each intake did not significantly change (FIG. 7g,h).

### 6) Monoamine Concentrations in the Brain

No significant differences in concentration of L-DOPA, dopamine and serotonin were found between the groups in any of the brain region studied (table 3).

TABLE 3
Concentrations of L-DOPA, dopamine and serotonin
between the groups the studied the brain regions.

| | | Control | LS + LG | Student's |
|---|---|---|---|---|
| | Mono | (pg/μg of | (pg/μg of | t-test, |
| Brain region | amine | tissue) | tissue) | p-value |
| Corpus | L-DOPA | 48.75 ± 9.72 | 53.21 ± 9.04 | 0.74 |
| striatum | DA | 1.08 ± 0.96 | 0.57 ± 0.27 | 0.54 |
| | 5-HT | 0.24 ± 0.11 | 0.44 ± 0.28 | 0.47 |
| Nucleus | L-DOPA | 36.98 ± 5.00 | 31.24 ± 4.53 | 0.4 |
| accumbens | DA | 1.93 ± 0.89 | 2.88 ± 1.25 | 0.54 |
| | 5-HT | 0.58 ± 0.25 | 0.92 ± 0.44 | 0.52 |
| Prefrontal | L-DOPA | 32.45 ± 5.71 | 35.25 ± 5.15 | 0.72 |
| cortex | DA | 6.51 ± 5.26 | 2.02 ± 0.74 | 0.54 |
| | 5-HT | 1.35 ± 0.93 | 0.18 ± 0.05 | 0.3 |
| Hippocampus | L-DOPA | 39.24 ± 5.50 | 61.55 ± 32.11 | 0.47 |
| | DA | 1.12 ± 0.68 | 2.40 ± 1.07 | 0.39 |
| | 5-HT | 0.45 ± 0.05 | 0.56 ± 0.19 | 0.76 |
| Amygdala | L-DOPA | 47.25 ± 2.66 | 44.32 ± 6.84 | 0.71 |
| | DA | 2.34 ± 0.93 | 1.54 ± 0.47 | 0.52 |
| | 5-HT | 0.41 ± 0.15 | 0.31 ± 0.07 | 0.6 |
| Hypothalamus | L-DOPA | 27.78 ± 7.09 | 29.86 ± 6.15 | 0.82 |
| | DA | 6.48 ± 4.21 | 7.49 ± 1.78 | 0.85 |
| | 5-HT | 1.97 ± 1.54 | 1.22 ± 0.59 | 0.60 |

### Example 4: Comparative OT-Like Immunoreactivity in Bacteria

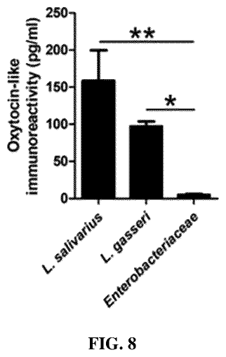

OT-like immunoreactivity was detected in total bacterial protein extracts from all 4 tested bacterial strains. It was found that OT-like concentrations in protein homogenates of both L. salivarius (158.7±41 μg/ml) and L. gasseri (97.2±13 μg/ml) were higher than that in Enterobacteriaceae (E. coli, H. alvei) (5.1±1 μg/ml, FIG. 8). For the reference, OT-like immunoreactivities of the mouse hypothalamus tissue and plasma, as measured by the same assay, were 471±0.1 pg/ml and 2.6±0.5 pg/ml, respectively.

### Brief Discussion of the Results

Oxytocin (OT) is a well-known modulator of motivated behavior with the main site of action in the brain. Peripheral OT signaling was also shown to be involved in the regulation of stress and behavior, ex. OT reduces activation of the hypothalamo-pituitary-adrenal (HPA) axis at its different levels, reducing ACTH and cortisol secretions (Legros JJ. Inhibitory effect of oxytocin on corticotrope function in humans: are vasopressin and oxytocin ying-yang neurohormones?Psychoneuroendocrinology. 2001; 26(7):649-55; Legros J J, Chiodera P, Geenen V. Inhibitory action of exogenous oxytocin on plasma cortisol in normal human subjects: Evidence of action at the adrenal level. Neuroendocrinology. 1988; 48(2):204-6.). Indeed, beside the brain, oxytocin receptors are present in several peripheral tissues. Of importance, OT receptors have been also found in the gut, located on the apical surface of enterocytes, i.e. directly accessible for gut bacteria-derived products (Welch M G, Tamir H, Gross K J, Chen J, Anwar M, Gershon M D. Expression and developmental regulation of oxytocin (OT) and oxytocin receptors (OTR) in the enteric nervous system (ENS) and intestinal epithelium. Journal of Comparative Neurology. 2009; 512(2):256-70). Without willing to be bound by a theory, OT-like and OT-like immunoreactive proteins (see example 4) produced by Lactobacilli may play a role of ligands for such receptors. Moreover, there is an inverse relation between plasma OT levels and depressive disorders. Several central and peripheral OT-targets may be responsible for OT anti-depressive effects, including the stimulation of monoamine production in the brain. However, in our study, we did not find significant differences in the monoamine levels measured in postmortem brain tissues (see example 3). Furthermore, the findings of Penagarikano et al. make it possible to use oxytocin and oxytocin-mediated pathways in autistic spectrum disorders (Penagarikano et al. Sci. Transl. Med. 2015; 7:271ra8)

The presented in vivo evidence shows that the provision of the LS and LG mix (Mix 1) according to the invention in mice improves their anxiety, CMS induced, relative depressive-like behavior. Following gavage, no significant differences in locomotor and anxiety-like behaviors were observed between the study groups in the open field, O-maze or the Phenotypic cages. However, during the CMS, the probiotic-treatment according to the invention reduced total immobility in both TST and FST and showed significantly increased escape energy in the TST as well as decreased immobility in the middle of the FST. The probiotic treated group also had a lower intake of 2% sucrose and a modified feeding pattern with a reduced body weight gain compared to the control.

Lastly, both studied Lactobacillus probiotic strains of LS and LG contain proteins with a high level of OT-like immunoreactivity. Thus, without willing to be bound by a theory, the effects of the presently claimed invention may be mediated by presenting an Oxytocin-like immunoreactivity (see the LS, LG bioactive peptides of example 1) and/or by OT-like immunoreactivity, thereby justifying the effects of the claimed invention in the treatment of OT-related diseases such as autism and improve anxiety related depression, and also reduce sugar craving, or binge-eating or even induce satiety particularly in chronically stressed subjects.

1. A method of treating autism and/or an anxiety related disorders selected from depression and an eating disorder in a subject in need thereof, comprising administering to the subject a therapeutically effective amount of a composition comprising a probiotic composition comprising a Lactobacillus salivarius strain and a Lactobacillus gasseri strain.

2. The method according to claim 1, wherein the probiotic composition presents an oxytocin-like immunoreactivity.

3. The method according to claim 1, wherein the probiotic composition stimulates the oxytocin receptor.

4. The method according to claim 1, wherein the anxiety related disorder is chronic mild stress.

5. The method according to claim 1, wherein the eating disorders are is selected from binge eating, sugar craving and anxiety related weight gain.

6. The method according to claim 1, wherein the probiotic composition consists of a Lactobacillus salivarius strain and a Lactobacillus gasseri strain.

7. The method according to claim 1, wherein the Lactobacillus salivarius strain is Lactobacillus salivarius DSM 16530 strain.

8. The method according to claim 1, wherein the probiotic composition expresses a bioactive peptide mixture comprising at least one, or at least four, or at least five bioactive peptides selected from the group consisting of peptides presenting from at least 80%, from at least 85%, from at least 90%, from at least 95%, or 100% identity with any one the peptides of SEQ ID 28, SEQ ID NO 29, SEQ ID NO 30, SEQ ID NO 33, SEQ ID NO 35, SEQ ID NO 36, and SEQ ID NO 37.

9. A composition comprising a probiotic composition consisting of a Lactobacillus salivarius strain and a Lactobacillus gasseri.

10. The composition according to claim 9, wherein the Lactobacillus salivarius strain is Lactobacillus salivarius DSM 16530 and/or wherein the Lactobacillus gasseri strain is Lactobacillus gasseri LMG P-29638.

11. The composition according to claim 9, wherein said composition further comprises at least one plant or plant extract, and/or at least one magnesium supplement; and/or at least one micronutrient.

12. The composition claim 9, wherein said composition is formulated in an oral dosage form selected from enterically-coated tablets and enterically-coated capsules, wherein the enterically-coated tablets and the enterically-coated capsules comprise an enteric-coating that is a mixture comprising hydroxypropyl methyl cellulose and gellan gum.

13. The composition according to claim 9, wherein said composition is formulated in a moisture-tight blister or container.

14. A method of treating autism or an anxiety related disorder selected from depression and an eating disorder in a subject in need thereof, comprising administering to the subject a therapeutic amount of a composition comprising a mixture of bioactive peptides comprising at least one bioactive peptide, wherein the at least one bioactive peptide has at least 80%, at least 85%, at least 90%, at least 95%, or 100% identity with any one of the peptides of SEQ ID 28, SEQ ID NO 29, SEQ ID NO 30, SEQ ID NO 33, SEQ ID NO 35, SEQ ID NO 36, and SEQ ID NO 37.

15. The method according to claim 14, wherein the mixture of bioactive peptides comprises at least one, or at least four, or at least five bioactive peptides, and wherein the at least one bioactive peptide is selected from the group consisting of peptides presenting from at least 80%, from at least 85%, from at least 90%, from at least 95%, or 100% identity with any one of the peptides of SEQ ID 28, SEQ ID NO 29, SEQ ID NO 30, SEQ ID NO 33, SEQ ID NO 35, SEQ ID NO 36, and SEQ ID NO 37.

16. The composition according to claim 11 wherein
the at least one plant or plant extract is selected from the group consisting of Rhodiola rosea, Withania somnifera, Melissa officinalis, Crocus sativus and Hypericum perforatum; and/or
the at least one magnesium supplement is magnesium bound to an amino acid or a magnesium salt selected from the group consisting of magnesium carbonate, magnesium oxide, magnesium acetate, magnesium ascorbate, magnesium citrate, magnesium gluconate, magnesium lactate, magnesium malate, magnesium pyrrolidone carboxylate, magnesium taurate, and magnesium threonate; and/or
the at least one micronutrient is selected from calcium, magnesium, phosphorus, iron, zinc, copper, iodine, selenium, beta carotene, Vitamin C, Vitamin B1, Vitamin B6, Vitamin B2, niacin, Vitamin B12, folic acid, biotin, Vitamin D, and Vitamin E.

17. The method of claim 14, wherein the composition comprises at least four bioactive peptides.
